(19) United States
(12) Patent Application Publication
(10) Pub. No.: US 2006/0162098 A1
(43) Pub. Date: Jul. 27, 2006

(54) Use of polycationic compounds for dyeing keratin fibres

(76) Inventor: Alain Lagrange, Coupvray (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/158,324

(22) Filed: Jun. 22, 2005

Related U.S. Application Data
(60) Provisional application No. 60/616,351, filed on Oct. 7, 2004.

(30) Foreign Application Priority Data
Jun. 23, 2004 (FR) 04 06851

(51) Int. Cl. A61K 8/00

(52) U.S. Cl. 8/405

(57) ABSTRACT

Use of polycationic compounds for dyeing keratin fibres The present patent application relates to the use, as direct dye in compositions for dyeing keratin fibres, in particular human keratin fibres such as the hair, or for the manufacture of such compositions, of a particular azo compound.

The present patent application relates to the use of particular polycationic compounds, as direct dyes in dye compositions for dyeing keratin fibres, and in particular human hair. The invention is also directed towards dye compositions based on these dyes, and also to the use of these compositions for dyeing keratin fibres.

It is known practice to dye keratin fibres, and in particular human hair, with dye compositions containing oxidation dye precursors, which are generally known as oxidation bases, such as ortho- or para-phenylenediamines, ortho- or para-aminophenols, and heterocyclic compounds. These oxidation bases are colourless or weakly coloured compounds that, when combined with oxidizing products, may give rise to coloured compounds by a process of oxidative condensation. These dyes, which are insoluble in the dyeing medium, are trapped inside the hair.

It is also known that the shades obtained with these oxidation bases may be varied by combining them with couplers or coloration modifiers, the latter being chosen especially from aromatic meta-diamines, meta-aminophenols, meta-diphenols and certain heterocyclic compounds such as indole compounds.

The variety of molecules used as oxidation bases and couplers allows a wide range of colours to be obtained.

The “permanent” coloration obtained using these oxidation dyes must moreover satisfy a certain number of requirements. Thus, it must have no toxicological drawback, it must be able to produce shades in the desired intensity, and it must show good resistance to external agents such as light, bad weather, washing, permanent-waving, perspiration and rubbing.

The dyes must also be able to cover grey hair and, finally, they must be as unselective as possible, i.e. they must produce the smallest possible differences in coloration along the same keratin fibre, which is generally differently sensitized (i.e. damaged) between its end and its root.

It is also known practice to dye keratin fibres by direct or semi-permanent dyeing. The process conventionally used in direct dyeing consists in applying to the keratin fibres direct dyes, which are coloured and colouring molecules that have affinity for the fibres, leaving these dyes on the fibres to allow the coloured molecules to penetrate into the hair by diffusion, and then rinsing the fibres.

In contrast with oxidation dye compositions, direct or semi-permanent dye compositions may be used without the presence of an oxidizing agent. These dyeing operations may be performed repeatedly without degrading the keratin fibre.

It is known practice, for example, to use nitrobenzene, anthraquinone, nitropyridine, azo, xanthene, acridine, azine or triarylmethane direct dyes.

This results in colorations that are particularly chromatic, but which are, however, temporary or semi-permanent on account of the nature of the bonds between the direct dyes and the keratin fibre. These interactions are such that the dyes are readily desorbed from the surface and/or the core of the fibre. The colorations generally show low dyeing power and poor fastness with respect to washing or perspiration. These direct dyes are also generally light-sensitive, since the chromophore has low resistance to photochemical attack, which leads to fading of the coloration of the hair over time. The light sensitivity of these dyes depends on their uniform distribution or distribution as aggregates in and/or on the keratin fibre.

Moreover, standard direct dyes are not always entirely harmless, which is why, in hair cosmetics, dyeing molecules of this type that are ever more efficient in terms of harmlessness are sought.

There is a real need for direct dye compositions that are improved in terms of shampoo fastness and uptake of the dye.

The Applicant has just discovered, surprisingly and advantageously, that the use of particular polycationic compounds in compositions for dyeing keratin fibres, in particular human keratin fibres such as the hair, make it possible to obtain dye compositions that have these improvements.

Besides their advantage in terms of harmlessness, the compositions according to the present patent application can produce dyeing results that are resistant to external attack (such as sunlight and bad weather) and also to shampoo and perspiration, and can produce strong and fast shades on the fibres.

One subject of the present invention is thus the use, as direct dye in compositions for dyeing keratin fibres, in particular human keratin fibres such as the hair, or for the manufacture of such compositions, of compounds of formula (I) described below.

A subject of the invention is also a dye composition for dyeing keratin fibres, in particular human keratin fibres such as the hair, comprising, in a suitable dyeing medium, at least one oxidation base and at least one compound of formula (I).

Another subject of the invention relates to a process for dyeing keratin fibres, in particular human keratin fibres such as the hair, using this composition.

A subject of the invention is also the use of the composition of the present invention on keratin fibres, in particular human keratin fibres such as the hair, to obtain dyeing results that show good resistance to external agents and to shampoo. The tautomeric forms of the compounds of formula (I) may also be used.

Other characteristics, aspects, subjects and advantages of the invention will emerge even more clearly on reading the description and the examples that follow.

The compounds that may be used as direct dyes according to the invention correspond to formula (I) 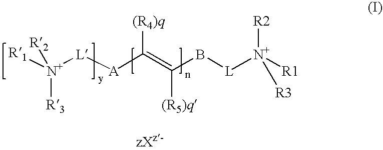
in which:

y denotes 0 or 1;

n denotes an integer ranging from 1 to 4;

q and q′ denote, independently of each other, 0 or 1;

X^{z′} denotes an organic or mineral anion;

z and z′ are such that the overall charge of the molecule is zero;

only one or both of the extreme carbon atoms of the group

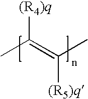

possibly forming part of the rings A and/or B;

A and/or B denotes(s), independently of each other, an aromatic non-heterocyclic cyclic group or a 5- to 15-membered, substituted or unsubstituted heterocyclic group, at least one of the groups A or B bearing at least one positive charge on condition that, if A is a non-heterocyclic ring, then B denotes a benzothiazolium, naphthothiazolium, naphthoxazolium, benzoxazolium, benzimidazolium, thiazolium, pyrimidobenzimidazolium, benzopyrroloimidazolium, pyrido benzoxazolium, pyrido benzothiazolium, pyrrolo benzoxazolium, pyrrolo benzothiazolium, naphthothiazolo pyridinium, azepino thiazolium, diazepino benzoxazolium, azepino benzoxazolium, pyrido benzoxazolium, azocino benzoxazolium, azocino benzothiazolium, azepino benzothiazolium or quinolinium group. Preferably A and B represent heterocycles;

L and L′ denote, independently of each other, a linear, branched or cyclic alkylene chain containing from 1 to 30 carbon atoms and/or a substituted or unsubstituted aromatic chain containing from 6 to 30 carbon atoms; the chain possibly being interrupted or ending with one or more heteroaromatic groups chosen from O, S, NH and NR where R represents an optionally substituted, linear or branched C1-C20 alkyl radical, or interrupted with one or more heterocyclic or non-heterocyclic, aromatic or non-aromatic cyclic radicals, the chain itself possibly bearing one or more quaternary cationic charges and one or more carbon atoms of the chain possibly being replaced with a carbonyl group;

R1, R2, R3, R′1, R′2 and R′3 denote, independently of each other, a linear, branched or cyclic, optionally substituted C1-C20 alkyl radical, two or more of these radicals possibly forming, with the nitrogen atom bearing them and optionally one of the carbon or nitrogen atoms of L for R1, R2 and/or R3 or L′ for R′1, R′2 and/or R′3, an optionally substituted saturated, unsaturated or aromatic heterocycle that may contain one or more additional hetero atoms; an optionally substituted C6-C30 aryl radical;

The radical borne by a quaternized or non-quaternized nitrogen atom engaged in an aromatic or non-aromatic, saturated or unsaturated heterocycle represents an optionally substituted, linear, branched or cyclic C1-C20 alkyl radical.

R, R4 and R5 denote, independently of each other, a hydrogen atom, a halogen atom, an optionally substituted C6-C30 aryl radical, or a linear, branched or cyclic, optionally substituted C1-C10 alkyl radical; the radicals R4 and R5 may form with the carbon atoms bearing them a 5- to 10-membered ring.

For the purposes of the present invention, the term “substituent” means a radical chosen from the following radicals: hydroxyl, amino, nitro, halogen, C1-C10 alkyl, C6-C30 aryl, C1-C10 hydroxyalkyl, mono(C1-C10)alkylamino or di(C1-C10)alkylamino which may optionally form, with the nitrogen atom to which each is linked, a saturated or unsaturated hereocycle, optionally comprising at least one other hetero atom, preferably nitrogen, mono- or di(C1-C10)hydroxyalkylamino, tri(C1-C10)alkylammonium, C1-C10 alkoxy optionally substituted by a tri(C1-C10)alkylammonium, amido, phenylamido, carboxyl in the form of an acid or a salt, (C1-C10)alkoxycarbonyl, sulfonato, (C1-C10)alkoxycarbonyl(C1-C10)alkyl, amido(C1-C10)alkylamido, sulfonyl difluoromethane, 5- to 10-membered heterocycle, C1-C10 alkyl heterocycle with a 5- to 10-membered heterocycle.

As heterocycles that are possible for A and B other than those mentioned above, mention may be made of imidazolium, pyridinium, triazolium, pyrazolium, pyrimidinium, indolium, indolinium and benzoselenazolium rings.

Non-cationic heterocycles that my be mentioned include non-cationic equivalents of the previously listed heterocycles, but also (3H) benzothiazolyl, (3H) benzoxazolyl, (2H) indolyl, (1H) quinolinyl, (1H) pyridinyl, thiazolidinyl, (4H) pyridinyl and (4H) pyrimidinyl.

Aromatic non-heterocyclic rings that may be mentioned include benzene, naphthalene and anthracene rings, these rings possibly being substituted with one or more of the substituents mentioned above.

Organic or mineral anions that may be mentioned include halides and in particular chloride, bromide or iodide ions, methanesulfonate ions, methosulfate ions, bromates or chlorate ions, acetate ions, sulfate ions, tartrate ions, lactate ions and citrate ions.

Among the compounds of formula (I) that may be mentioned are the compounds hereinbelow. The nature of the counterion X^{z-} is not critical. 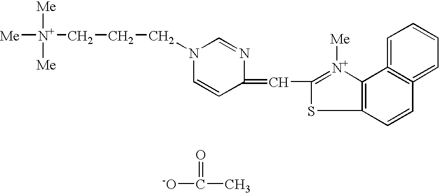
trimethyl{3-[4-(1-methylnaphtho[1,2-d]thiazolium-2-ylmethylene)-4H-pyrimidin-1-yl]propyl}ammonium acetate 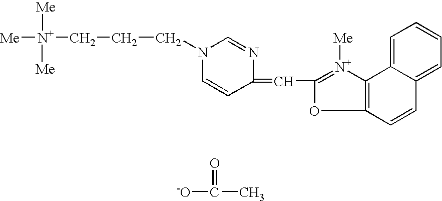
trimethyl{3-[4-(1-methylnaphtho[1,2-d]oxazolium-2-ylmethylene)-4H-pyrimidin-1-yl]propyl}ammonium acetate 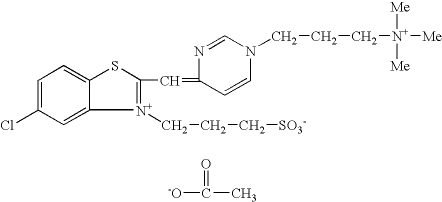
(3-{4-[5-chloro-3-(3-sulfopropyl)benzothiazolium-2-ylmethylene]-4H-pyrimidin-1-yl]propyl}ammonium acetate 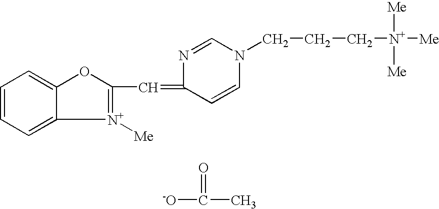
trimethyl{3-[4-(3-methylbenzoxazolium-2-ylmethylene)-4H-pyrimidin-1-yl]propyl}ammonium acetate 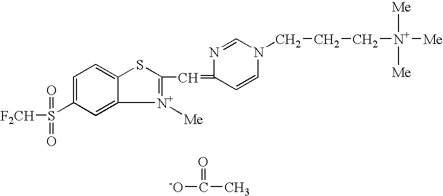
(3-{4-[5-(difluoromethanesulfonyl)-3-methylbenzothiazol-2-ylmethylene]-4H-pyrimidin-1-yl]propyl}ammonium acetate 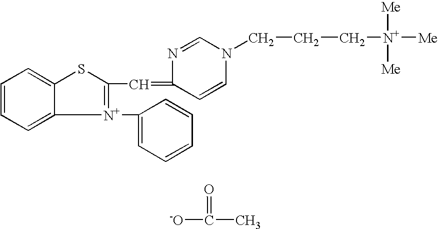
trimethyl{3-[4-(3-phenylbenzothiazol-2-ylmethylene)-4H-pyrimidin-1-yl]propyl}ammonium acetate 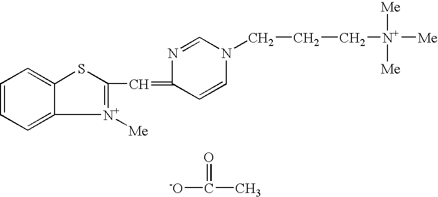
trimethyl{3-[4-(3-methylbenzothiazolium-2-ylmethylene)-4H-pyrimidin-1-yl]propyl}ammonium acetate 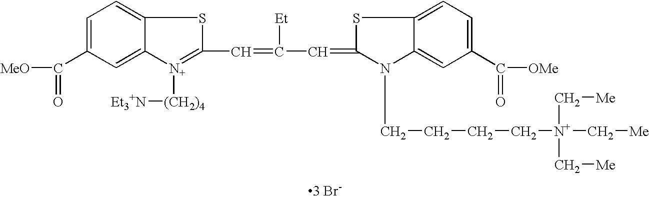
5-(methoxycarbonyl)-2-[2-[[5-(methoxycarbonyl)-3-[4-(triethylammonio)butyl]-2(3H)benzothiazolylidene]methyl]-1-butenyl]-3-[4triethylammonio)butyl]benzothiazolium tribromide 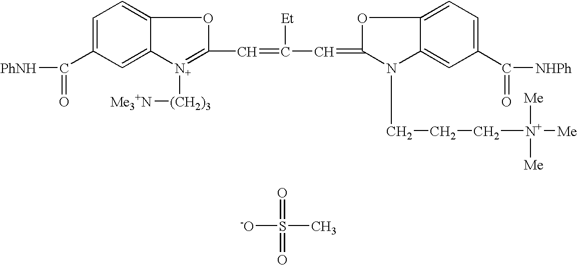
5-[(phenylamino)carbonyl]-2-[2-[[5-[(phenylamino)carbonyl]-3-[3-(trimethylammonio)propyl]-2(3H)benzoxazolylidene]methyl]-1-butenyl]-3-[3-(trimethylammonio)propyl]benzoxazolium tris(methanesulfonate) 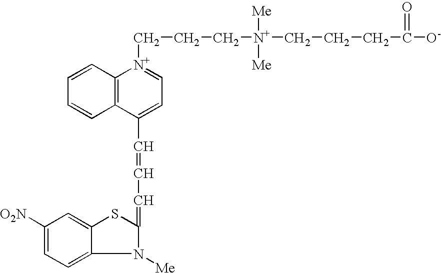
Quinolinium, 1-[3-[(3-carboxypropyl)dimethylammonio]propyl]-4-[3-(3-methyl-6-nitro-2(3H)benzothiazolylidene)-1-propenyl]-, mono(inner salt) (9CI) 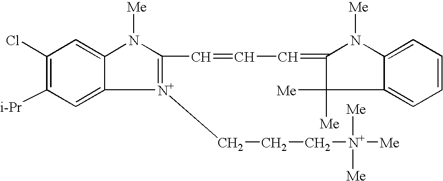
1H-Benzimidazolium, 5-chloro-2-[3-(1,3-dihydro-1,3,3-trimethyl-2H-indol-2-ylidene)-1-propenyl]-3-methyl-6-(1-methylethyl)-1-[3-(trimethylammonio)propyl]-(9CI) 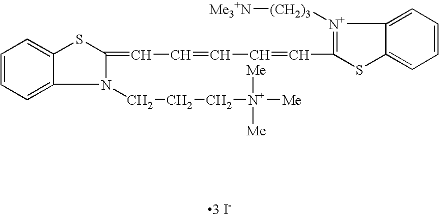
3-[3-(trimethylammonio)propyl]-2-[5-[3-[3-(trimethylammonio)-propyl]-2(3H)benzothiazolylidene]-1,3-pentadienyl]benzothiazolium triiodide 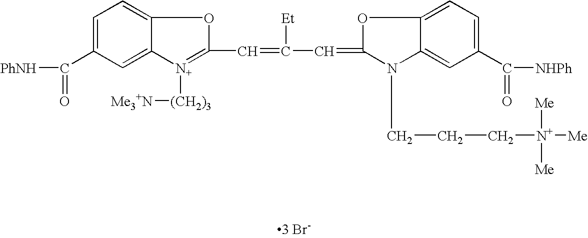
5-[(phenylamino)carbonyl]-2-[2-[[5-[(phenylamino)carbonyl]-3-[3-(trimethylammonio)propyl]-2(3H)benzoxazolylidene]methyl]-1-butenyl]-3-[3-(trimethylammonio)propyl]benzoxazolium tribromide 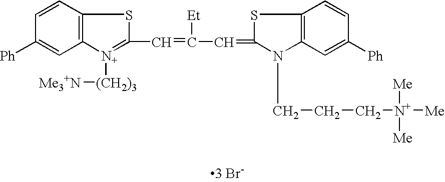
5-phenyl-2-[2-[[5-phenyl-3-[3-(trimethylammonio)propyl]-2(3H)-benzothiazolylidene]methyl]-1-butenyl]-3-[3-(trimethylammonio)-propyl]benzothiazolium tribromide 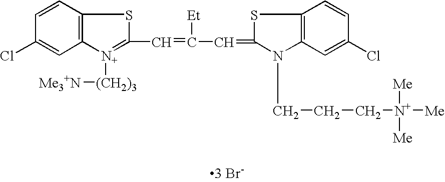
5-chloro-2-[2-[[5-chloro-3-[3-(trimethylammonio)propyl]-2(3H)benzo-thiazolylidene]methyl]-1-butenyl]-3-[3-(trimethylammonio)propyl]-benzothiazolium tribromide 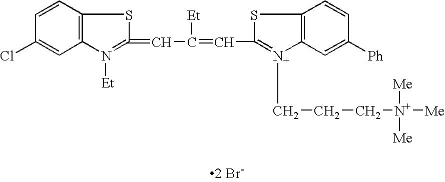
2-[2-[(5-chloro-3-ethyl-2(3H)benzothiazolylidene)methyl]-1-butenyl]-5-phenyl-3-[3-(trimethylammonio)propyl]benzothiazolium dibromide 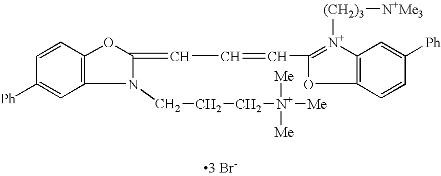
5-phenyl-2-[3-[5-phenyl-3-[3-(trimethylammonio)propyl]-2(3H)benzoxazolylidene]-1-propenyl]-3-[3-(trimethylammonio)propyl]benzoxazolium tribromide 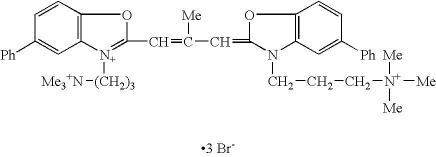
2-[2-methyl-3-[5-phenyl-3-[3-(trimethylammonio)propyl]-2(3H)benzoxazolylidene]-1-propenyl]-5-phenyl-3-[3-(trimethylammonio)-propyl]benzoxazolium tribromide 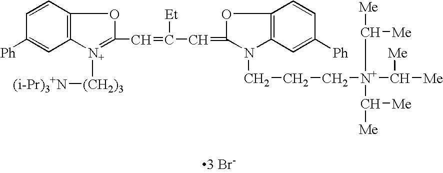
5-phenyl-2-[2-[[5-phenyl-3-[3-[tris(1-methylethyl)ammonio]propyl]-2(3H)benzoxazolylidene]methyl]-1-butenyl]-3-[3-[tris(1-methylethyl)-ammonio]propyl]benzoxazolium tribromide 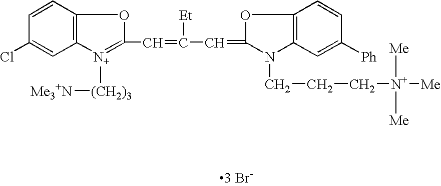
5-chloro-2-[2-[[5-phenyl-3-[3-(trimethylammonio)propyl]-2(3H)benzoxazolylidene]methyl]-1-butenyl]-3-[3-(trimethylammonio)propyl]-benzoxazolium tribromide 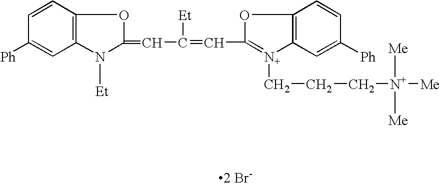
2-[2-[(3-ethyl-5-phenyl-2(3H)benzoxazolylidene)methyl]- 1-butenyl]-5-phenyl-3-[3-(trimethylammonio)propyl]benzoxazolium dibromide 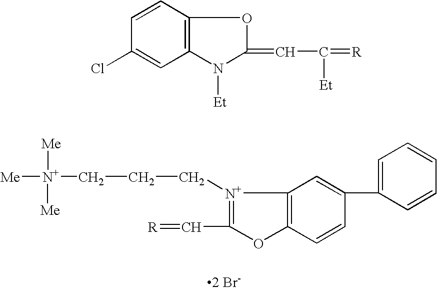
2-[2-[(5-chloro-3-ethyl-2(3H)benzoxazolylidene)methyl]-1-butenyl]-5-phenyl-3-[3-(trimethylammonio)propyl]benzoxazolium dibromide 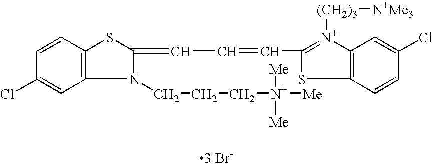
5-chloro-2-[3-[5-chloro-3-[3-(trimethylammonio)propyl]-2(3H)benzothiazolylidene]-1-propenyl]-3-[3-(trimethylammonio)propyl]benzothiazolium tribromide 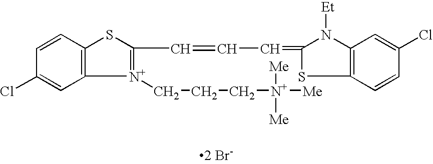
5-chloro-2-[3-(5-chloro-3-ethyl-2(3H)benzothiazolylidene)-1-propenyl]-3-[3-(trimethylammonio)propyl]benzothiazolium dibromide 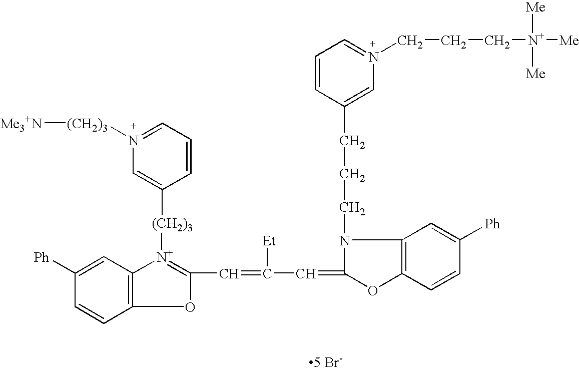
5-phenyl-2-[2-[[5-phenyl-3-[3-[1-[3-(trimethylammonio)propyl]pyridinium-3-yl]propyl]-2(3H)benzoxazolylidene]methyl]-1-butenyl]-3-[3-[1-[3-(trimethylammonio)propyl]pyridinium-3-yl]propyl]benzoxazolium pentabromide 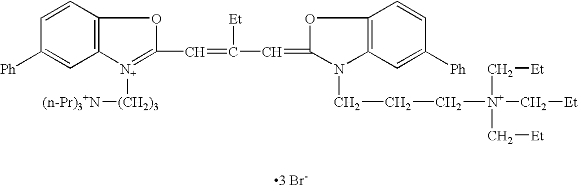
5-phenyl-2-[2-[[5-phenyl-3-[3-(tripropylammonio)propyl]-2(3H)benzoxazolylidene]methyl]-1-butenyl]-3-[3-(tripropylammonio)propyl]-benzoxazolium tribromide 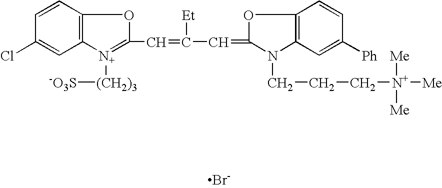
5-chloro-2-[2-[[5-phenyl-3-[3-(trimethylammonio)propyl]-2(3H)benzoxazolylidene]methyl]-1-butenyl]-3-(3-sulfopropyl)benzoxazolium bromide 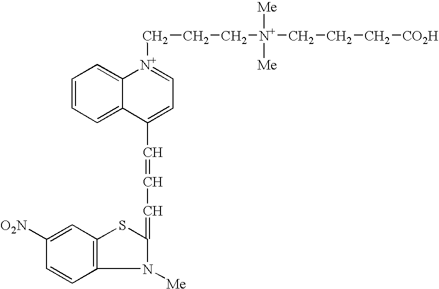
1-[3-[(3-carboxypropyl)dimethylammonio]propyl]-4-[3-(3-methyl-6-nitro-2(3H)benzothiazolylidene)-1-propenyl]quinolinium 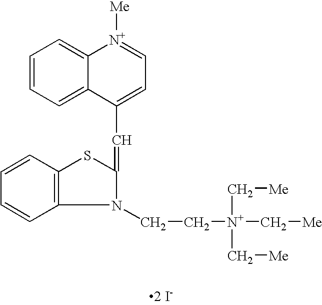
1-methyl-4-[[3-[2-(triethylammonio)ethyl]-2(3H)benzothiazolylidene]-methyl]quinolinium diiodide 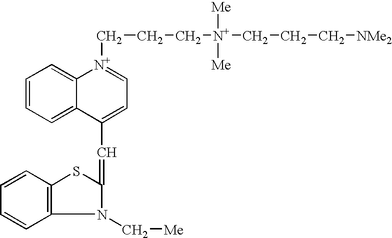
1[3-[[3-(dimethylamino)propyl]dimethylammonio]propyl]-4-[(3-ethyl-2(3H)benzothiazolylidene)methyl]quinolinium 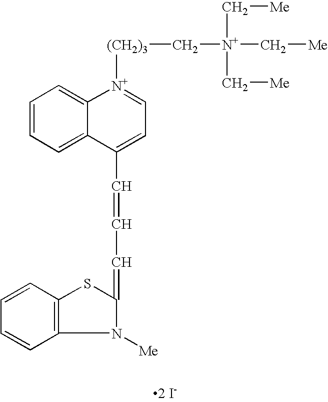
4-[3-(3-methyl-2(3H)benzothiazolylidene)-1-propenyl]-1-[4-(triethyl-ammonio)butyl]quinolinium diiodide 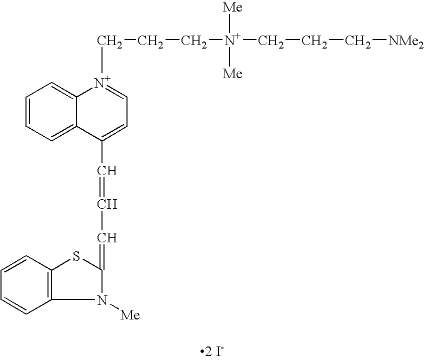
1-[3-[[3-(dimethylamino)propyl]dimethylammonio]propyl]-4-[3-(3-methyl-2(3H)benzothiazolylidene)-1-propenyl]quinolinium diiodide 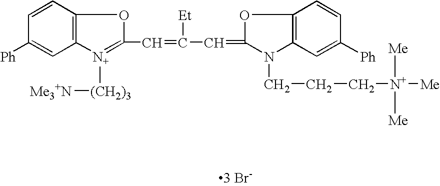
5-phenyl-2-[2-[[5-phenyl-3-[3-(trimethylammonio)propyl]-2(3H)benzoxazolylidene]methyl]-1-butenyl]-3-[3-(trimethylammonio)propyl]-benzoxazolium tribromide 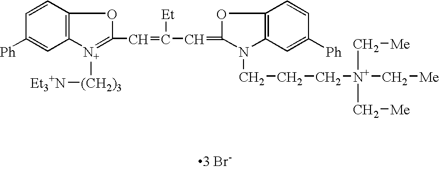
5-phenyl-2-[2-[[5-phenyl-3-[3-(triethylammonio)propyl]-2(3H)benzoxazolylidene]methyl]-1-butenyl]-3-[3-(triethylammonio)propyl]benzoxazolium tribromide 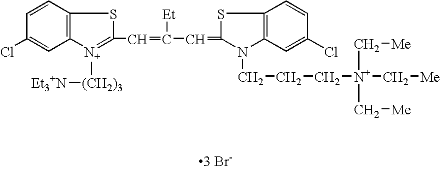
5-chloro-2-[2-[[5-chloro-3-[3-(triethylammonio)propyl]-2(3H)benzothiazolylidene]methyl]-1-butenyl]-3-[3-(triethylammonio)propyl]-benzothiazolium tribromide 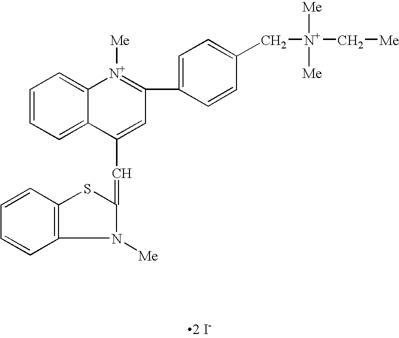
2-[4-[(ethyldimethylammonio)methyl]phenyl]-1-methyl-4-[(3-methyl-2(3H)benzothiazolylidene)methyl]quinolinium diiodide 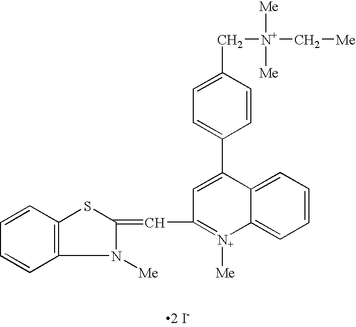
4-[4-[(ethyldimethylammonio)methyl]phenyl]-1-methyl-2-[(3-methyl-2(3H)benzothiazolylidene)methyl]quinolinium diiodide 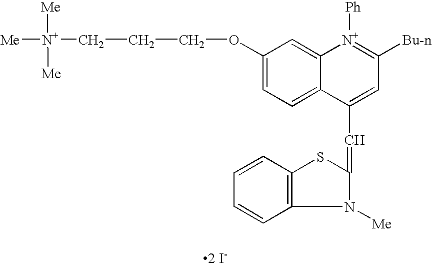
2-butyl-4-[(3-methyl-2(3H)benzothiazolylidene)methyl]-1-phenyl-7-[3-(trimethylammonio)propoxy]quinolinium diiodide 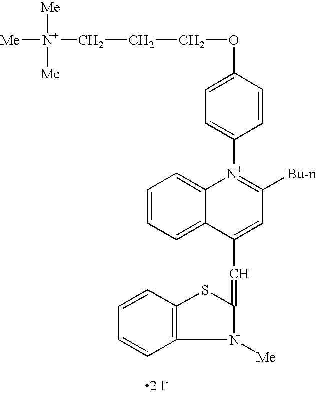
2-butyl-4-[3-methyl-2(3H)benzolylidene)methyl]-1-[4-[3-(trimethylammonio)propoxy]phenyl]quinolinium diiodide 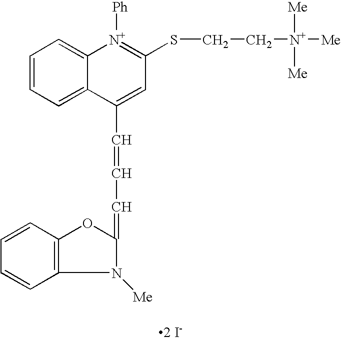
4-[3-(3-methyl-2(3H)benzoxazolylidene)-1-propenyl]-1-phenyl-2-[[2-(trimethylammonio)ethyl]thio]quilinium diiodide 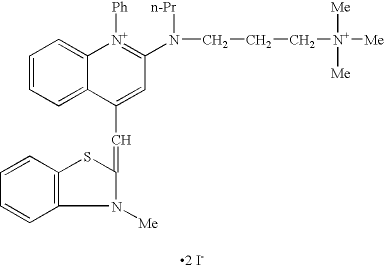
4-[(3-methyl-2(3H)benzothiazolylidene)methyl]-1-phenyl-2-[propyl[3-(trimethylammonio)propyl]amino]quinolinium diiodide 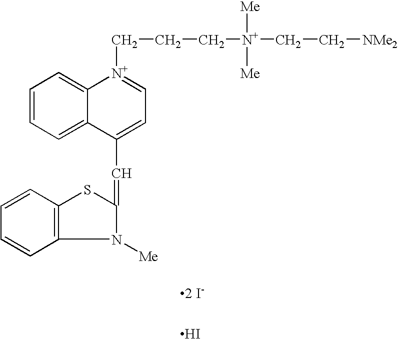
1-[3-[[2-(dimethylamino)ethyl]dimethylammonio]propyl]-4-[(3-methyl-2(3h)benzothiazolylidene)methyl]quinolinium diiodide, monohydriodide 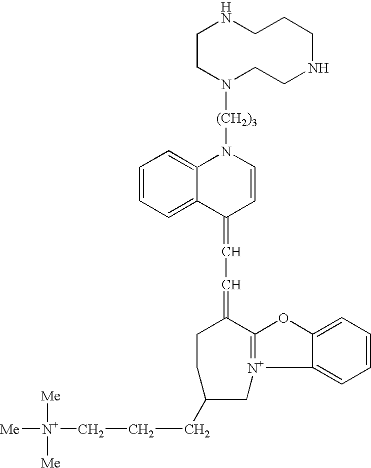
7,8,9,10-tetrahydro-6-[[1-[3-(octahydro-1,4,7-triazecin-4(1H)yl)-propyl]-4(1H)quinolinylidene]ethylidene]-9-[3-(trimethylammonio)-propyl]-6H-azepino[2,1-b]benzoxazolium 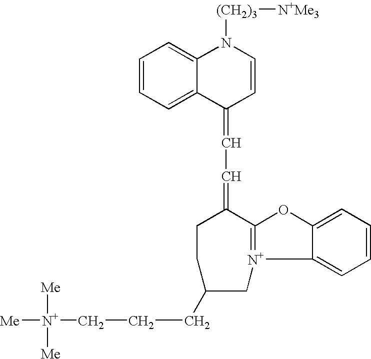
7,8,9,10-tetrahydro-9-[3-(trimethylammonio)propyl]-6-[[1-[3-(trimethylammonio)propyl]-4(1H)quinolinylidene]ethylidene]-6H-azepino[2,1-b]benzoxazolium 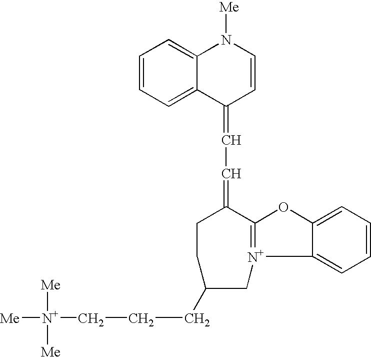
7,8,9,10-tetrahydro-6-[(1-methyl-4(1H)quinolinylidene)ethylidene]-9-[3-(trimethylammonio)propyl]-6H-azepino[2,1 -b]benzoxazolium 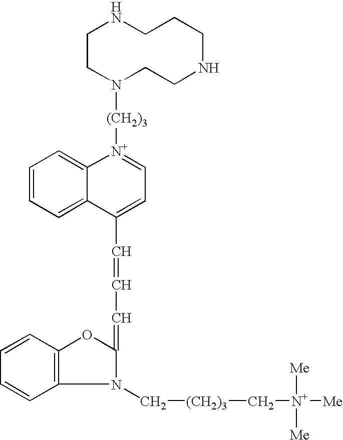
1-[3-(octahydro-1,4,7-triazecin-4(1H)yl)propyl]-4-[3-[3 -[5-(trimethyl-ammonio)pentyl]-2(3H)benzoxazolylidene]-1-propenyl]quinolinium 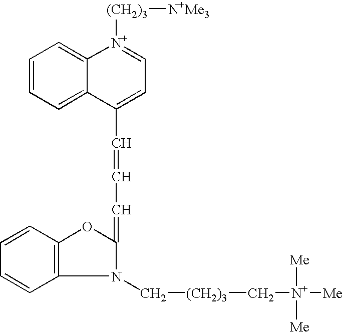
quinolinium, 1-[3-(trimethylammonio)propyl]-4-[3-[3-[5-(trimethyl-ammonio)pentyl]-2(3H)benzoxazolylidene]-1-propenyl]- (9CI) 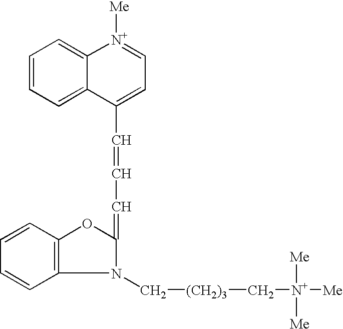
1-methyl-4-[3-[3-[5-(trimethylammonio)pentyl]-2(3H)benzoxazolylidene]-1-propenyl]quinolinium 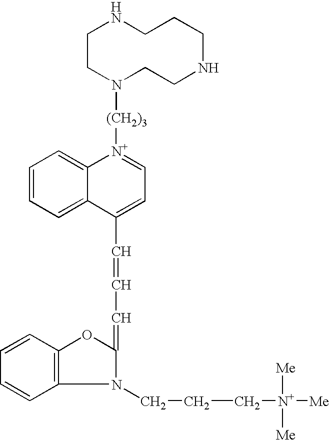
1-[3-(octahydro-1,4,7-triazecin-4(1H)ylidene)propyl]-4-[3-[3-[3-(trimethylammonio)propyl]-2(3H)benzoxazolylidene]-1-propenyl]-quinolinium 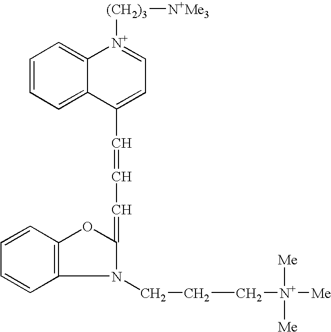
1-[3-(trimethylammonio)propyl]-4-[3-[3-[3-(trimethylammonio)-propyl]-2(3H)benzoxazolylidene]-1-propenyl]quinolinium 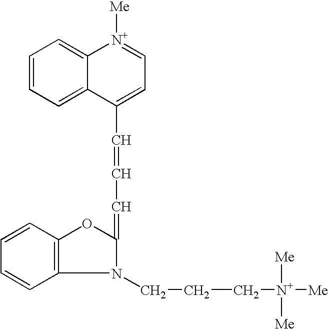
1-methyl-4-[3 -[3-[3-(trimethylammonio)propyl]-2(3H)benzoxazolylidene]-1-propenyl]quinolinium 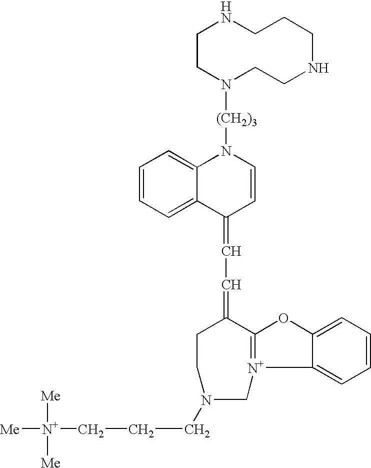
2,3,4,5-tetrahydro-5-[[1-[3-(octahydro-1,4,7-triazecin-4(1H)yl)-propyl]-4(1H)quinolinylidene]ethylidene]-2-[3-(trimethylammonio)-propyl]-1H-[1,3]diazepino[7,1-b]benzoxazol-11-ium 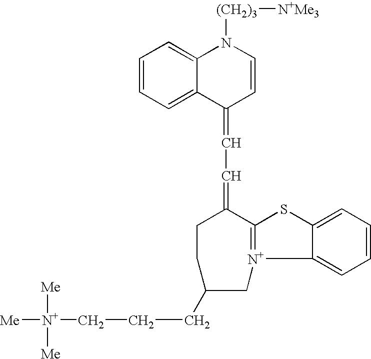
7,8,9,10-tetrahydro-9-[3-(trimethylammonio)propyl]-6-[[1-[3-(trimethylammonio)propyl]-4(1H)quinolinylidene]ethylidene]-6H-azepino[2,1-b]benzothiazolium 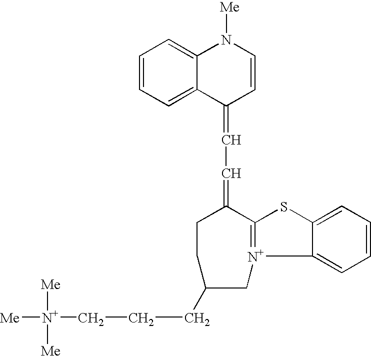
7,8,9,10-tetrahydro-6-[(1-methyl-4(1H)quinolinylidene)ethylidene]-9-[3-(trimethylammonio)propyl]-6H-azepino[2,1-b]benzothiazolium 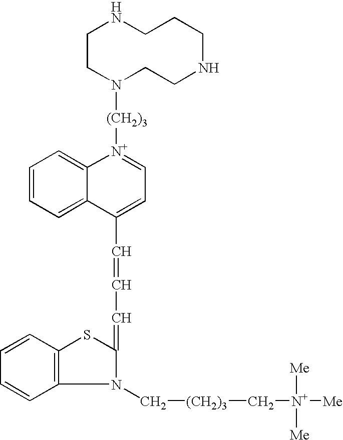
1-[3-(octahydro-1,4,7-triazecin-4(1H)yl)propyl]-4-[3-[3-[5-(trimethyl-ammonio)pentyl]-2(3H)benzothiazolylidene]-1-propenyl]quinolinium 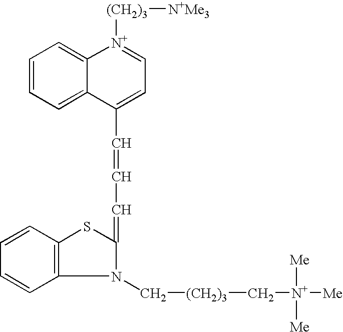
1[3-(trimethylammonio)propyl]-4-[3-[3-[5-(trimethylammonio)-pentyl]-2(3H)benzothiazolylidene]-1-propenyl]quinolinium 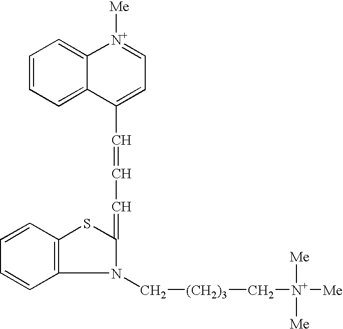
1-methyl-4-[3-[3-[5-(trimethylammonio)pentyl]-2(3H)benzothiazolylidene]-1-propenyl]quinolinium 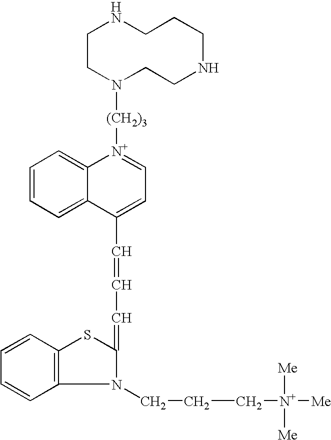
1-[3-(octahydro-1,4,7-triazecin-4(1H)ylidene)propyl]-4-[3-[3-[3-(trimethylammonio)propyl]-2(3H)benzothiazolylidene]-1-propenyl]-quinolinium 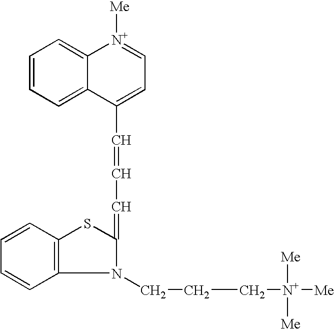
1-methyl-4-[3-[3-[3-(trimethylammonio)propyl]-2(3H)benzothiazolylidene]-1-propenyl]quinolinium 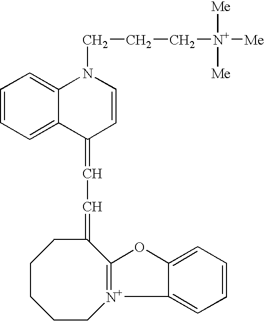
6,7,8,9,10,11-hexahydro-6-[[1-[3-(trimethylammonio)propyl]-4(1H)-quinolinylidene]ethylidene]azocino[2,1-b]benzoxazolium 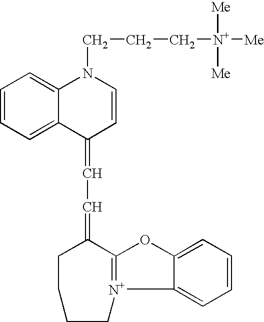
7,8,9,10-tetrahydro-6-[[1-[3-(trimethylammonio)propyl]-4(1H)quinolinylidene]ethylidene]-6H-azepino[2,1-b]benzoxazolium 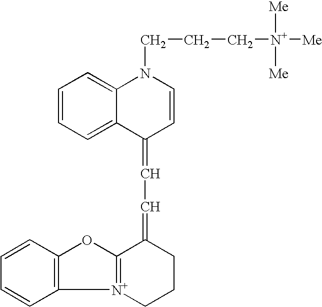
1,2,3,4-tetrahydro-4-[[1-[3-(trimethylammonio)propyl]-4(1H)quinolinylidene]ethylidene]pyrido[2,1-b]benzoxazolium 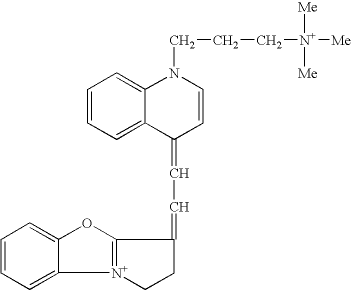
2,3-dihydro-3-[[1-[3-(trimethylammonio)propyl]-4(1H)quinolinylidene]ethylidene]-1H-pyrrolo[2,1-b]benzoxazolium 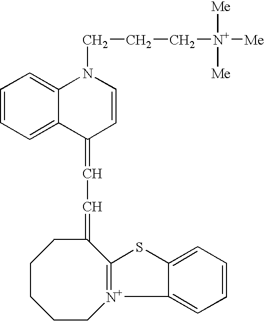
6,7,8,9,10,11-hexahydro-6-[[1-[3-(trimethylammonio)propyl]-4(1H)-quinolinylidene]ethylidene]azocino[2,1-b]benzothiazolium 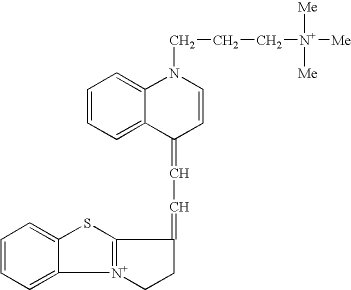
2,3-dihydro-3-[[1-[3-(trimethylammonio)propyl]-4(1H)quinolinylidene]ethylidene]-1H-pyrrolo[2,1-b]benzothiazolium 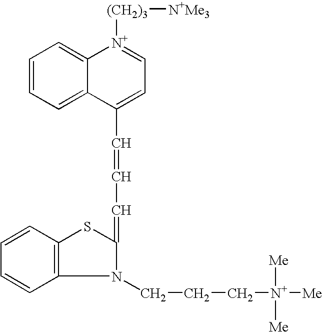
1-[3-(trimethylammonio)propyl]-4-[3-[3-[3-(trimethylammonio)-propyl]-2(3H)benzothiazolylidene]-1-propenyl]quinolinium 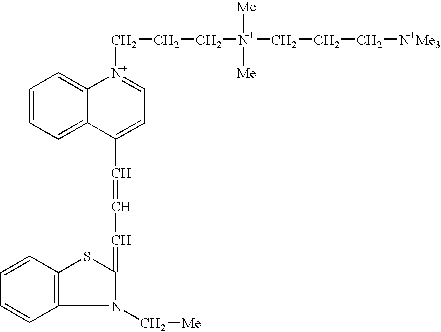
1-[3-[dimethyl[3-(trimethylammonio)propyl]ammonio]propyl]-4-[3-(3-ethyl-2(3H)benzothiazolylidene)-1-propenyl]quinolinium 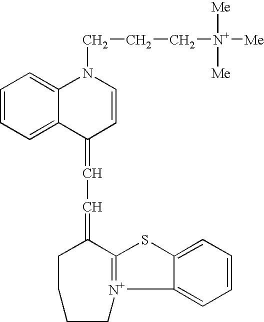
7,8,9,10-tetrahydro-6-[[1-[3-(trimethylammonio)propyl]-4(1H)quinolinylidene]ethylidene]-6H-azepino[2,1-b]benzothiazolium 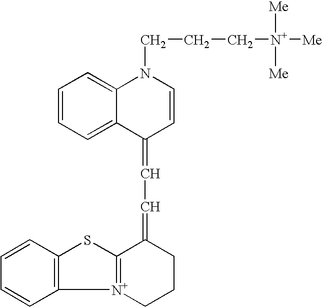
1,2,3,4-tetrahydro-4-[[1-[3-(trimethylammonio)propyl]-4(1H)quinolinylidene]ethylidene]pyrido[2,1-b]benzothiazolium 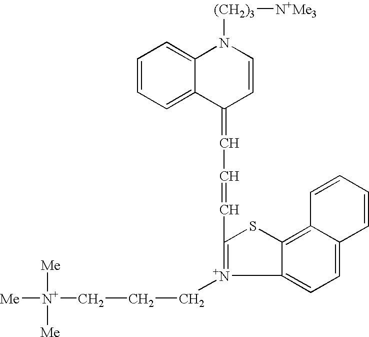
3-[3-(trimethylammonio)propyl]-2-[3-[1-[3-(trimethylammonio)-propyl]-4(1H)quinolinylidene]-1-propenyl]naphtho[2,1-d]thiazolium 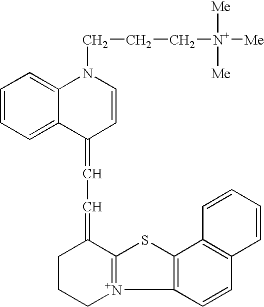
8,9,10,11-tetrahydro-11-[[1-[3-(trimethylammonio)propyl]-4(1H)-quinolinylidene]ethylidene]naphtho[2′,1′:4,5]thiazolo[3,2-a]pyridinium 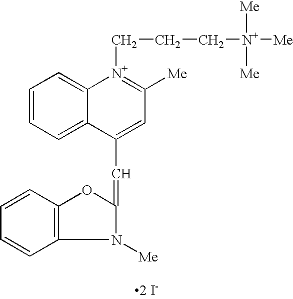
2-methyl-4-[(3-methyl-2(3H)benzoxazolylidene)methyl]-1-[3-(trimethylammonio)propyl]quinolinium diiodide 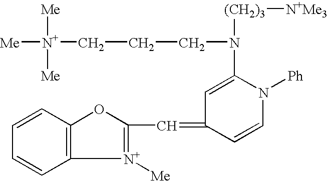
2-[[2-[bis[3-(trimethylammonio)propyl]amino]-1-phenyl-4(1H)pyridinylidene]methyl]-3-methylbenzoxazolium 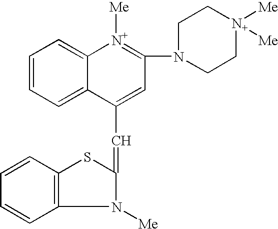
2-(4,4-dimethylpiperazinium-1-yl)-1-methyl-4-[(3-methyl-2(3H)benzothiazolylidene)methyl]quinolinium 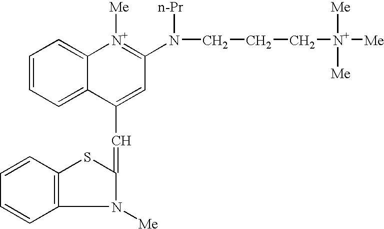
1-methyl-4-[(3-methyl-2(3H)benzothiazolylidene)methyl]-2-[propyl[3-(trimethylammonio)propyl]amino]quinolinium 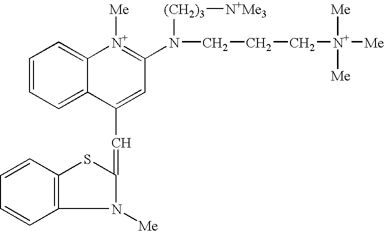
2-[bis[3-(trimethylammonio)propyl]amino]-1-methyl-4-[(3-methyl-2(3H)benzothiazolylidene)methyl]quinolinium 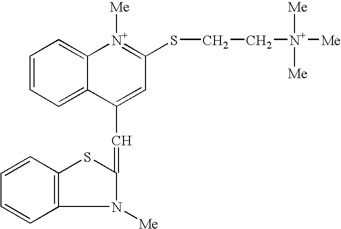
1-methyl-4-[(3-methyl-2(3H)benzothiazolylidene)methyl]-2-[[2-(trimethylammonio)ethyl]thio]quinolinium 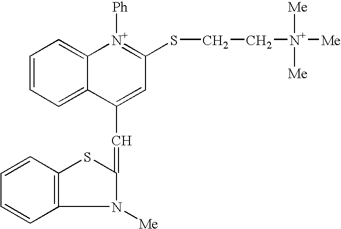
4-[(3-methyl-2(3H)benzothiazolylidene)methyl]-1-phenyl-2-[[2-(trimethylammonio)ethyl]thio]quinolinium 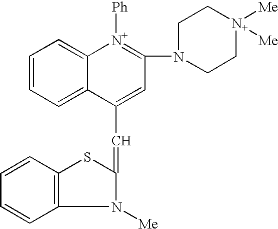
2-(4,4-dimethylpiperazinium-1-yl)-4-[(3-methyl-2(3H)benzothiazolylidene)methyl]-1-phenylquinolinium 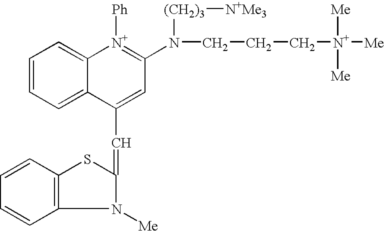
2- [bis[3-(trimethylammonio)propyl]amino]-4-[(3-methyl-2(3H)benzothiazolylidene)methyl]-1-phenylquinolinium 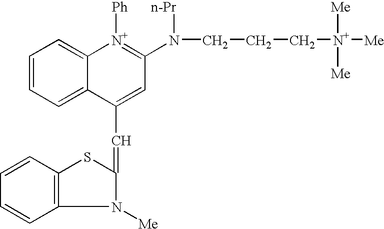
4-[(3-methyl-2(3H)benzothiazolylidene)methyl]-1-phenyl-2-[propyl[3-(trimethylammonio)propyl]amino]quinolinium 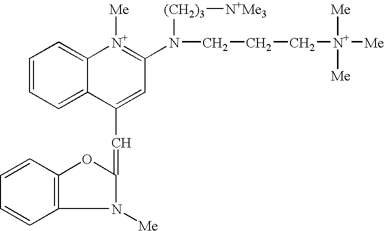
2-[bis[3-(trimethylammonio)propyl]amino]-1-methyl-4-[(3-methyl-2(3H)benzoxazolylidene)methyl]quinolinium 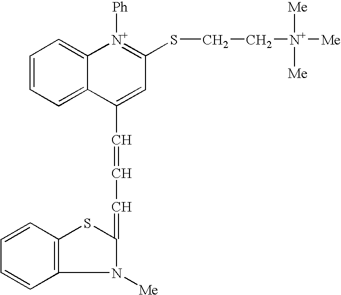
4-[3-(3-methyl-2(3H)benzothiazolylidene)-1-propenyl]-1-phenyl-2-[[2-(trimethylammonio)ethyl]thio]quinolinium 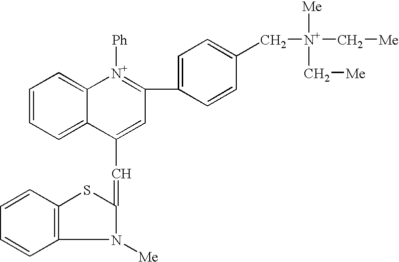
2-[4-[(diethylmethylammonio)methyl]phenyl]-4-[(3-methyl-2(3H)-benzothiazolylidene)methyl]-1-phenylquinolinium 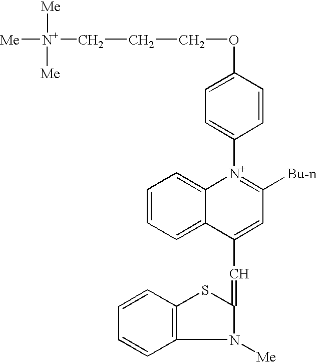
2-butyl-4-[(3-methyl-2(3H)benzothiazolylidene)methyl]-1-[4-[3-(trimethylammonio)propoxy]phenyl]quinolinium 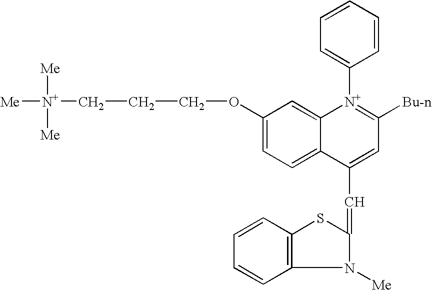
2-butyl-4-[(3-methyl-2(3H)benzothiazolylidene)methyl]-1-phenyl-7-[3-(trimethylammonio)propoxy]quinolinium 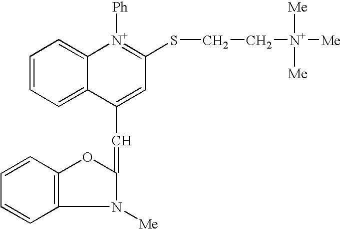
4-[(3-methyl-2(3H)benzoxazolylidene)methyl]-1-phenyl-2-[[2-(trimethylammonio)ethyl]thio]quinolinium 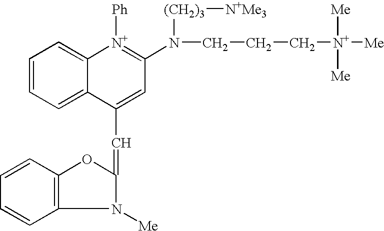
2-[bis[3-(trimethylammonio)propyl]amino]-4-[(3-methyl-2(3H)benzoxazolylidene)methyl]-1-phenylquinolinium 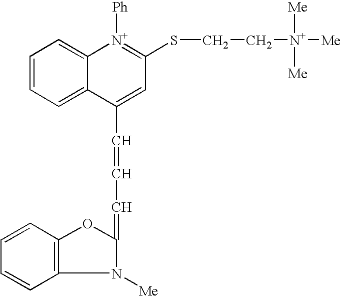
4-[3-(3-methyl-2(3H)benzoxazolylidene)-1-propenyl]-1-phenyl-2-[[2-(trimethylammonio)ethyl]thio]quinolinium 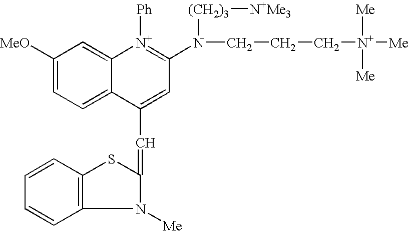
2-[bis[3-(trimethylammonio)propyl]amino]-7-methoxy-4-[(3-methyl-2(3H)benzothiazolylidene)methyl]-1-phenylquinolinium 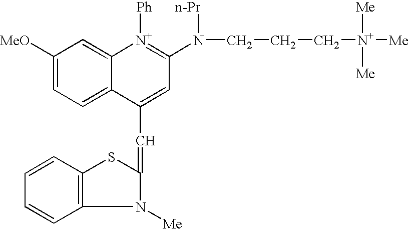
7-methoxy-4-[(3-methyl-2(3H)benzothiazolylidene)methyl]-1-phenyl-2-[propyl[3-(trimethylammonio)propyl]amino]quinolinium 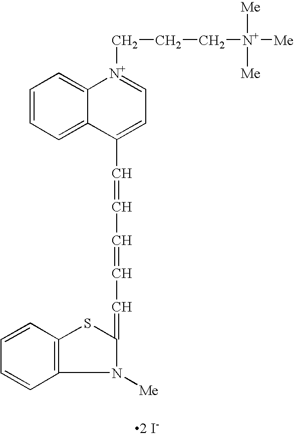
quinolinium, 4-[5-(3-methyl-2(3H)benzothiazolylidene)-1,3-pentadienyl]-1-[3-(trimethylammonio)propyl], diiodide (9CI) (3-methyl-2-[5-[1-[3-(trimethylammonio)propyl]-4(1H)quinolinylidene]-1,3-pentadienyl]benzothiazolium diiodide) 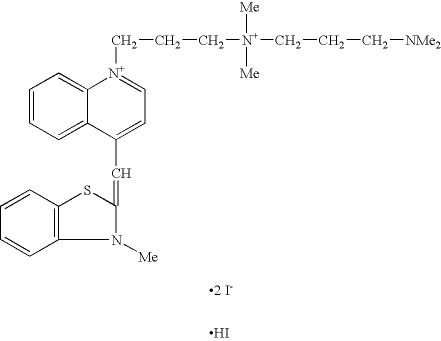
1-[3-[[3-(dimethylamino)propyl]dimethylammonio]propyl]-4-[(3-methyl-2(3H)benzothiazolylidene)methyl]quinolinium diiodide, monohydriodide 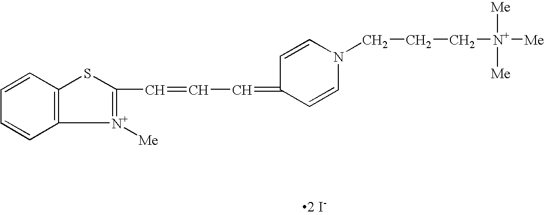
3-methyl-2-[3-[1-[3-(trimethylammonio)propyl]-4(1H)pyridinylidene]-1-propenyl]benzothiazolium diiodide 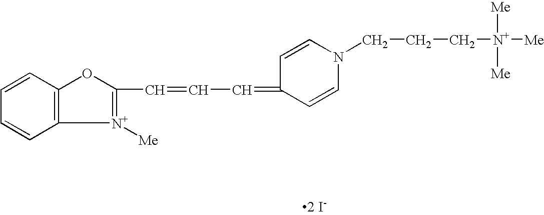
3-methyl-2-[3-[1-[3-(trimethylammonio)propyl]-4(1H)pyridinylidene]-1-propenyl]benzoxazolium diiodide 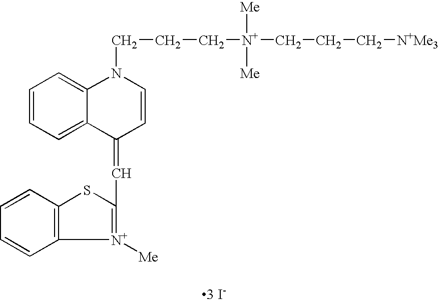
2-[[1-[3-[dimethyl[3-(trimethylammonio)propyl]ammonio]propyl]-4(1H)quinolinylidene]methyl]-3-methylbenzothiazolium triiodide 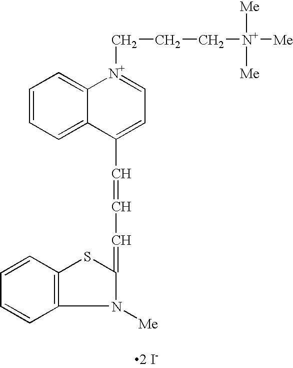
4-[3-(3-methyl-2(3H)benzothiazolylidene)-1-propenyl]-1-[3-(trimethylammonio)propyl]quinolinium diiodide 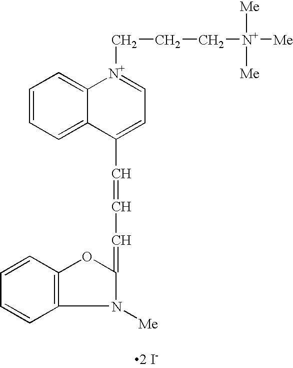
4-[3-(3-methyl-2(3H)benzoxazolylidene)-1-propenyl]-1-[3-(trimethylammonio)propyl]quinolinium diiodide 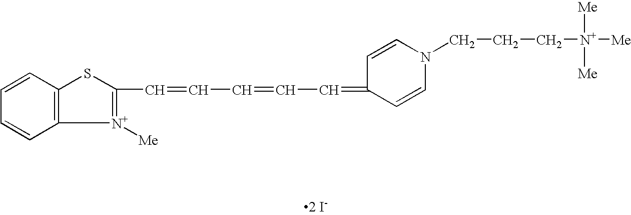
3-methyl-2-[5-[1-[3-(trimethylammonio)propyl]-4(1H)pyridinylidene]-1,3-pentadienyl]benzothiazolium diiodide 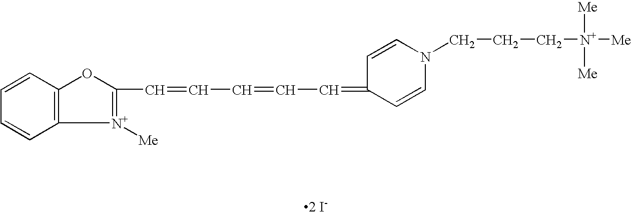
3-methyl-2-[5-[1-[3-(trimethylammonio)propyl]-4(1H)pyridinylidene]-1,3-pentadienyl]benzoxazolium diiodide 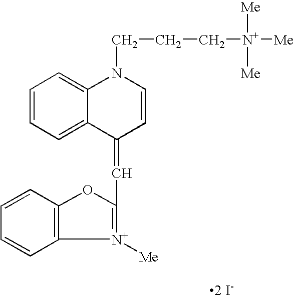
3-methyl-2-[[1-[3-(trimethylammonio)propyl]-4(1H)quinolinylidene]-methyl]benzoxazolium diiodide 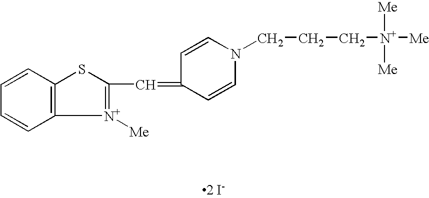
3-methyl-2-[[1-[3-(trimethylammonio)propyl]-4(1H)pyridinylidene]-methyl]benzothiazolium diiodide 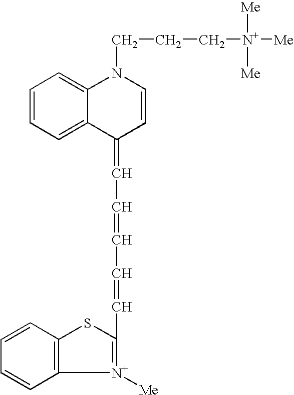
3-methyl-2-[5-[1-[3-(trimethylammonio)propyl]-4(1H)quinolinylidene]-1,3-pentadienyl]benzothiazolium 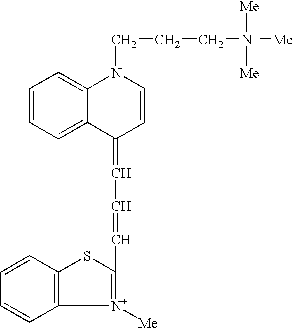
3-methyl-2-[3-[1-[3-(trimethylammonio)propyl]-4(1H)quinolinylidene]-1-propenyl]benzothiazolium 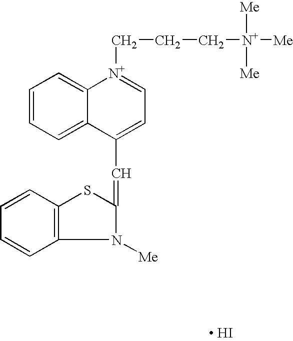
4-[(3-methyl-2(3H)benzothiazolylidene)methyl]-1-[3-(trimethylammonio)propyl]quinolinium 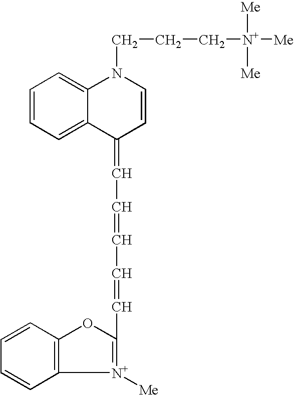
3-methyl-2-[5-[1-[3-(trimethylammonio)propyl]-4(1H)quinolinylidene]-1,3-pentadienyl]benzoxazolium 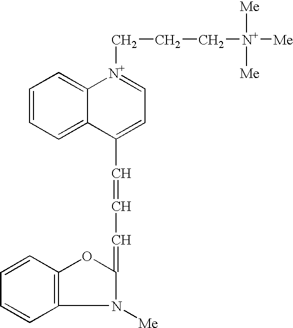
4-[3-(3-methyl-2(3H)benzoxazolylidene)-1-propenyl]-1-[3-(trimethylammonio)propyl]quinolinium 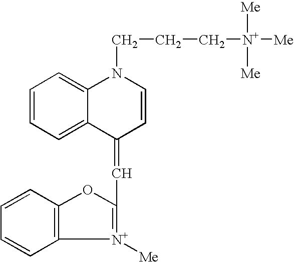
3-methyl-2-[[1-[3-(trimethylammonio)propyl]-4(1H)quinolinylidene]-methyl]benzoxazolium 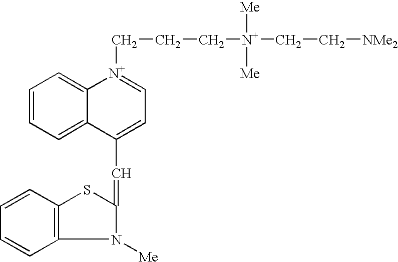
1-[3-[[2-(dimethylamino)ethyl]dimethylammonio]propyl]-4-[(3-methyl-2(3H)benzothiazolylidene)methyl]quinolinium 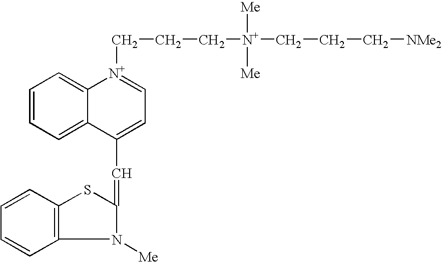
1-[3-[[3-(dimethylamino)propyl]dimethylammonio]propyl]-4-[(3-methyl-2(3H)benzothiazolylidene)methyl]quinolinium 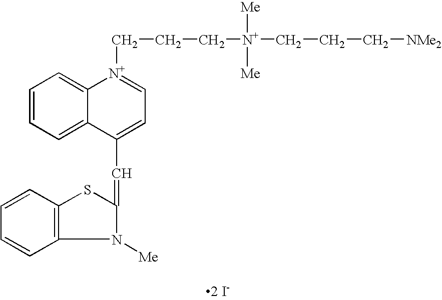
1-[3-[[3-(dimethylamino)propyl]dimethylammonio]propyl]-4-[(3-methyl-2(3H)benzothiazolylidene)methyl]quinolinium diiodide 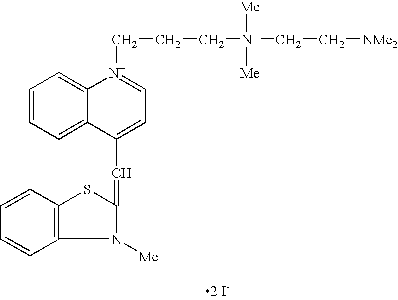
1-[3-[[2-(dimethylamino)ethyl]dimethylammonio]propyl]-4-[(3-methyl-2(3H)benzothiazolylidene)methyl]quinolinium diiodide 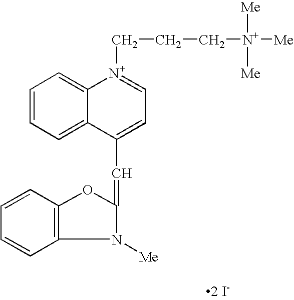
4-[(3-methyl-2(3H)benzoxazolylidene)methyl]-1-[3-(trimethyl-ammonio)propyl]quinolinium diiodide 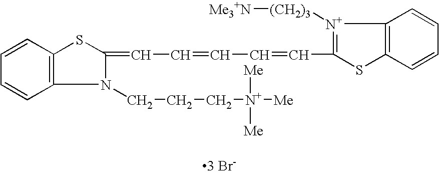
3-[3-(trimethylammonio)propyl]-2-[5-[3-[3-(trimethylammonio)-propyl]-2(3H)benzothiazolylidene]-1,3-pentadienyl]benzothiazolium tribromide 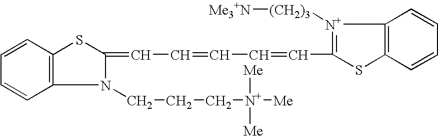
3-[3-(trimethylammonio)propyl]-2-[5-[3-[3-(trimethylammonio)-propyl]-2(3H)benzothiazolylidene]-1,3-pentadienyl]benzothiazolium 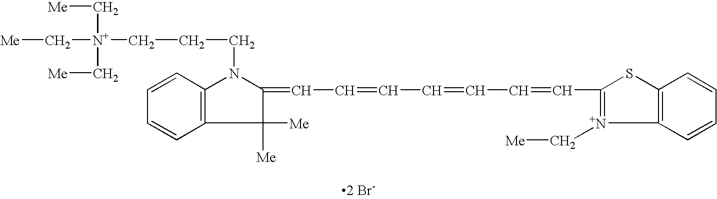
2-[7-[1,3-dihydro-3,3-dimethyl-1-[3-(triethylammonio)propyl]-2H-indol-2-ylidene]-1,3,5-heptatrienyl]-3-ethylbenzothiazolium dibromide 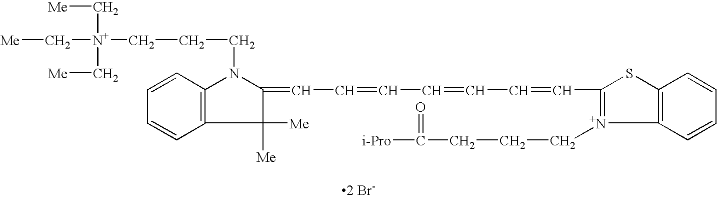
2-[7-[1,3-dihydro-3,3-dimethyl-1-[3-(triethylammonio)propyl]-2H-indol-2-ylidene]-1,3,5-heptatrienyl]-3-[4-(1-methylethoxy)-4-oxo-butyl]benzothiazolium dibromide 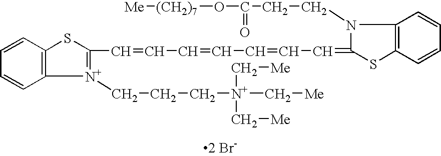
2-[7-[3-[3-(octyloxy)-3-oxopropyl]-2(3H)benzothiazolylidene]-1,3,5-heptatrienyl]-3-[3-(triethylammonio)propyl]benzothiazolium dibromide 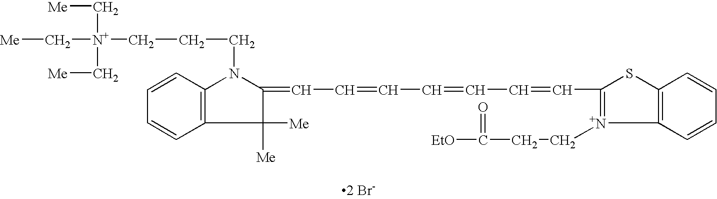
2-[7-[1,3-dihydro-3,3-dimethyl-1-[3-(triethylammonio)propyl]-2H-indol-2-ylidene]-1,3,5-heptatrienyl]-3-(3-ethoxy-3-oxopropyl)benzothiazolium dibromide 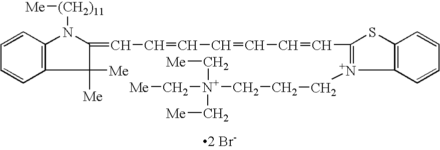
2-[7-(1-dodecyl-1,3-dihydro-3,3-dimethyl-2H-indol-2-ylidene)-1,3,5-heptatrienyl]-3-[3-(triethylammonio)propyl]benzothiazolium dibromide 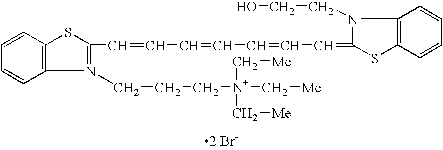
2-[7-[3-(2-hydroxyethyl)-2(3H)benzothiazolylidene]-1,3,5-hepta-trienyl]-3-[3-(triethylammonio)propyl]benzothiazolium dibromide 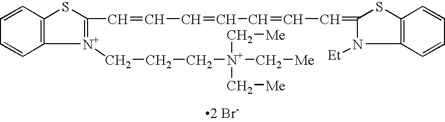
2-[7-(3-ethyl-2(3H)benzothiazolylidene)-1,3,5-heptatrienyl]-3-[3-(triethylammonio)propyl]benzothiazolium dibromide 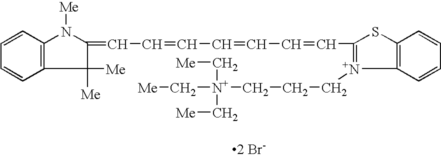
2-[7-(1,3-dihydro-1,3,3-trimethyl-2H-indol-2-ylidene)-1,3,5-heptatrienyl]-3-[3-(triethylammonio)propyl]benzothiazolium dibromide 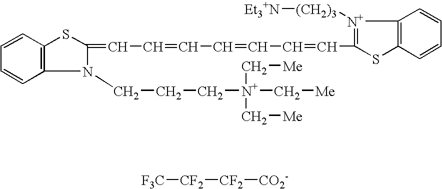
benzothiazolium, 3-[3-(triethylammonio)propyl]-2-[7-[3-[3-(triethylammonio)propyl]-2(3H)benzothiazolylidene]-1,3,5-heptatrienyl]-, salt with heptafluorobutanoic acid (1:3) (9CI)
(butanoic acid, heptafluoro-, ion(1-),3-[3-(triethylammonio)propyl]-2-[7-[3-[3-(triethylammonio)propyl]-2(3H)benzothiazolylidene]-1,3,5-heptatrienyl]benzothiazolium) 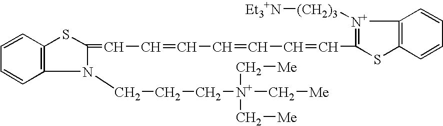
3-[3-(triethylammonio)propyl]-2-[7-[3-[3-(triethylammonio)propyl]-2(3H)benzothiazolylidene]-1,3,5-heptatrienyl]benzothiazolium 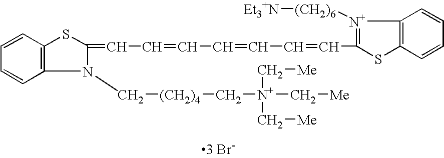
3-[6-(triethylammonio)hexyl]-2-[7-[3-[6-(triethylammonio)hexyl]-2(3H)benzothiazolylidene]-1,3,5-heptatrienyl]benzothiazolium tribromide 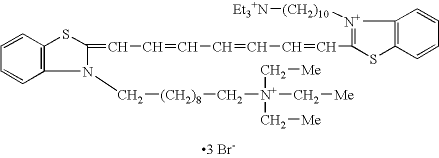
3-[10-(triethylammonio)decyl]-2-[7-[3-[10-(triethylammonio)decyl]-2(3H)benzothiazolylidene]-1,3,5-heptatrienyl]benzothiazolium tribromide 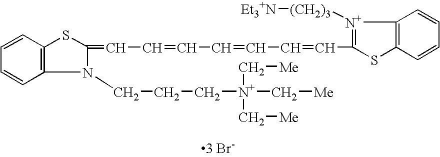
3-[3-(triethylammonio)propyl]-2-[7-[3-[3-(triethylammonio)propyl]-2(3H)benzothiazolylidene]-1,3,5-heptatrienyl]benzothiazolium tribromide 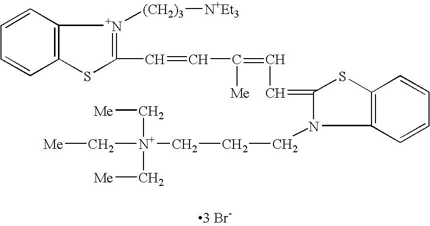
2-[3-methy-5-[3-[3-(triethylammonio)propyl]-2(3H)benzothiazolylidene]-1,3-pentadienyl]-3-[3-(triethylammonio)propyl]benzothiazolium tribromide 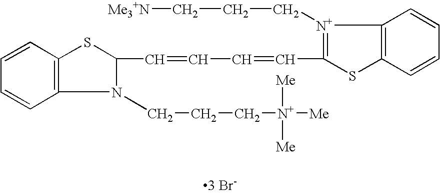
2-[4-[2,3-dihydro-3-[3-(trimethylammonio)propyl]-2-benzothiazolyl]-1,3-butadienyl]-3-[3-(trimethylammonio)propyl]benzothiazolium tribromide 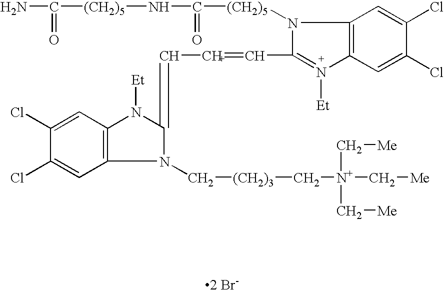
1-[6-[(6-amino-6-oxohexyl)amino]-6-oxohexyl]-5,6-dichloro-2-[3-[5,6-dichloro-1-ethyl-1,3-dihydro-3-[5-(triethylammonio)pentyl]-2H-benzimidazol-2-ylidene]-1-propenyl]-3-ethyl-1H-benzimidazolium dibromide 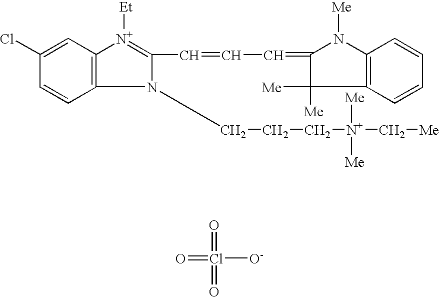
5-chioro-1-(3-dimethylaminopropyl)-3-ethyl-2,3-dihydro-2-[3-(1,3,3-trimethyl-2-indolinylidene)propenyl]-2-benzimidazolocarbonium perchlorate perchlorate 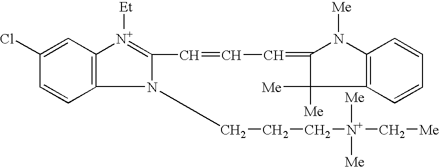
5-chloro-2-[3-(1,3-dihydro-1,3,3-trimethyl-2H-indol-2-ylidene)-1-propenyl]-3-ethyl-1-[3-(ethyldimethylammonio)propyl]-1H-benzimidazolium 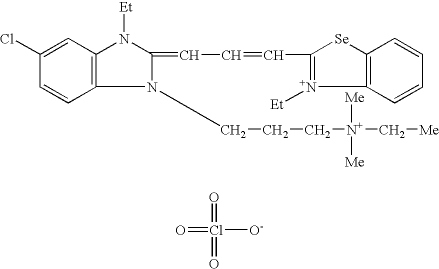
5-chloro-1-(3-dimethylaminopropyl)-3-ethyl-2-[3-(3-ethyl-2-benzoselenazolinylidene)propenyl]-2,3-dihydro-2-benzimidazolocarbonium perchlorate (6CI) 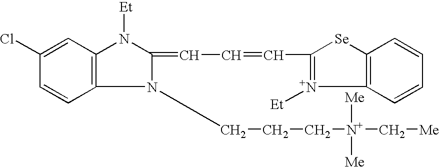
2-[3-[5-chloro-3-ethyl-1-[3-(ethyldimethylammonio)propyl]-1,3-dihydro-2H-benzimidazol-2-ylidene]-1-propenyl]-3-ethylbenzoselenazolium 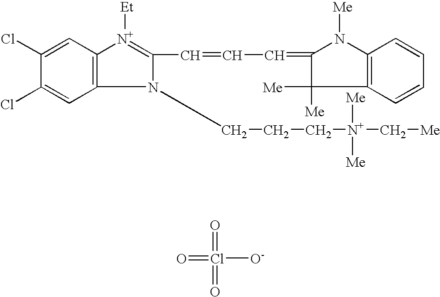
5,6-dichloro-1-(3-dimethylaminopropyl)-3-ethyl-2,3-dhydro-2-[3-(1,3,3-trimethyl-2-indolinylidene)propenyl]-2-benzimidazolocarbonium perchlorate ethoperchlorate (6CI)
(2-[3-[5,6-dichloro-1-(3-dimethylaminopropyl)-3-ethyl-2-benzimidazolinylidene]propenyl]-2,3-dihydro-1,3,3-trimethyl-2-indolocarbonium perchlorate) 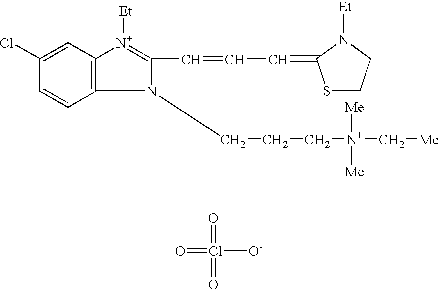
5-chloro-1-(3-dimethylaminopropyl)-3-ethyl-2-[3-(3-ethyl-2-thiazolidinylidene)propenyl]-2,3-dihydro-2-benzimidazolocarbonium perchlorate (6CI) 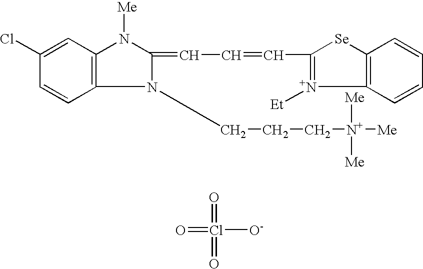
1-(3-mimethylaminopropyl)-2-[3-(3-ethyl-2-benzoselenazolinylidene)-propenyl]-2,3-dihydro-3-methyl-2-benzimidazolocarbonium perchlorate perchlorate (6CI) 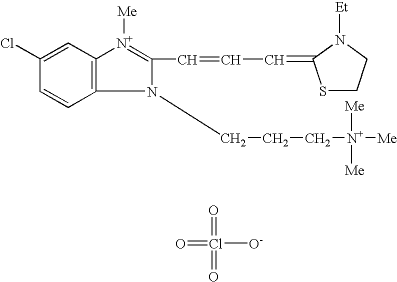
5-chloro-1-(3-dimethylaminopropyl)-2-[3-(3-ethyl-2-thiazolidinylidene)propenyl]-2,3-dihydro-3-methyl-2-benzimidazolocarbonium perchlorate perchlorate (6CI) 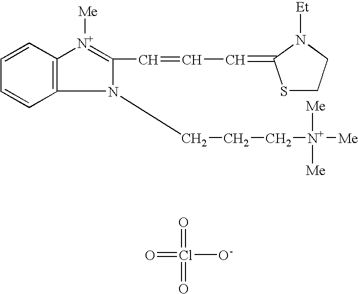
1-(3-dimethylaminopropyl)-2-[3-(3-ethyl-2-thiazolidinylidene)-propenyl]-2,3-dihydro-3-methyl-2-benzimidazolocarbonium perchlorate (6CI) 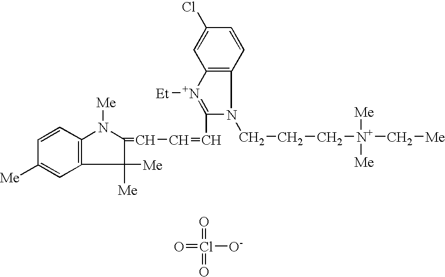
2-[3-[5-chloro-1-(3-dimethylaminopropyl)-3-ethyl-2-benzimidazolinylidene]propenyl]-2,3-dihydro-1,3,3,5-tetramethyl-2-indolocarbonium perchlorate (6CI) 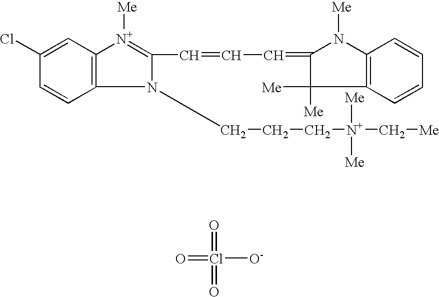
1H-benzimidazolium, 5-chloro-2-[3-(1,3-dihydro-1,3,3-trimethyl-2H-indol-2-ylidene)-1-propenyl]-3-methyl-1-[3-(trimethylammonio)-propyl]-, diperchlorate (9CI)
(2-[3-[5-chloro-1-(3-dimethylaminopropyl)-3-methyl-2-benzimidazolinylidene]propenyl]-2,3-dihydro-1,3,3-trimethyl-2-indolocarbonium perchlorate) 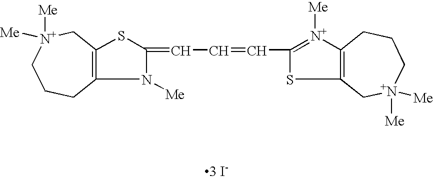
5,6,7,8-tetrahydro-1,5,5-trimethyl-2-[3-(5,6,7,8-tetrahydro-1,5,5-trimethyl-4H-thiazolo[5,4-c]azepin-2(1H)ylidene)propenyl]-4H-thiazolo[5,4-c]azepinediium triiodide 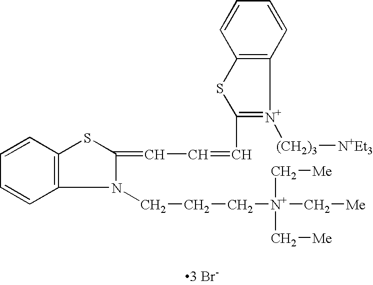
3-[3-(triethylammonio)propyl]-2-[3-[3-[3-(triethylammonio)propyl]-2(3H)benzothiazolylidene]-1-propenyl]benzothiazolium tribromide 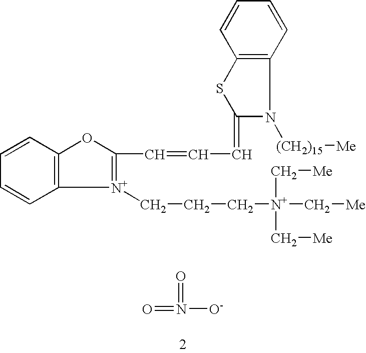
2-[3-(3-hexadecyl-2(3H)benzothiazolylidene)-1-propenyl]-3-[3-(triethylammonio)propyl]benzozazolium dinitrate 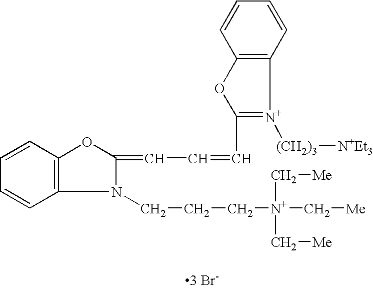
3-[3-(triethylammonio)propyl]-2-[3-[3-[3-(triethylammonio)propyl]-2(3H)benzoxazolylidene]-1-propenyl]benzoxazolium tribromide 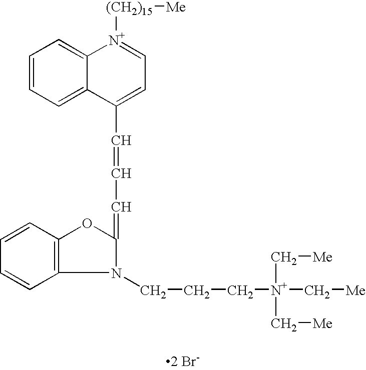
1-hexadecyl-4-[3-[3-[3-(triethylammonio)propyl]-2(3H)benzoxazolylidene]-1-propenyl]quinolinium dibromide 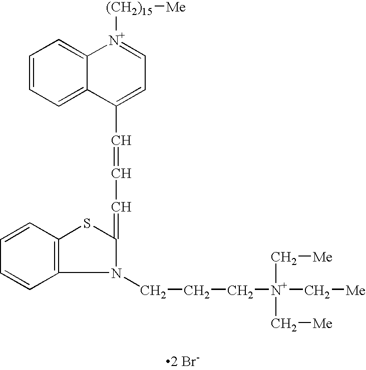
1-hexadecyl-4-[3-[3-[3-(triethylammonio)propyl]-2(3H)benzothiazolylidene]-1-propenyl]quinolinium dibromide 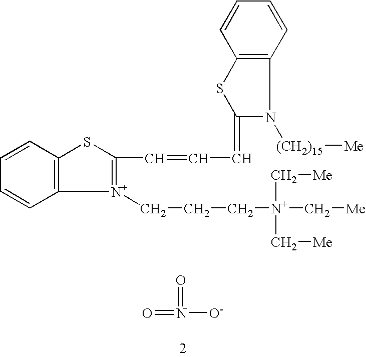
2-[3-(3-hexadecyl-2(3H)benzothiazolylidene)-1-propenyl]-3-[3-(triethylammonio)propyl]benzothiazolium dinitrate 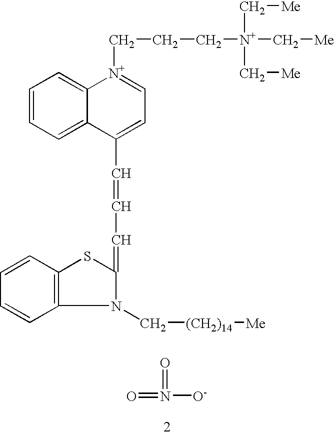
4-[3-(3-hexadecyl-2(3H)benzothiazolylidene)-1-propenyl]-1-[3-(triethylammonio)propyl]quinolinium dinitrate 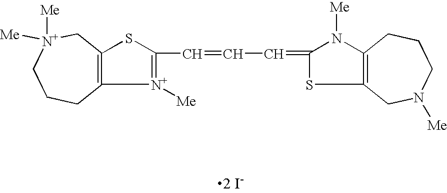
2-[3-(1,4,5,6,7,8-hexahydro-1,5-dimethyl-2H-thiazolo[5,4-c]azepin-2-ylidene)propenyl]-5,6,7,8-tetrahydro-1,5,5-trimethyl-4H-thiazolo [5,4-c]azepinium diiodide 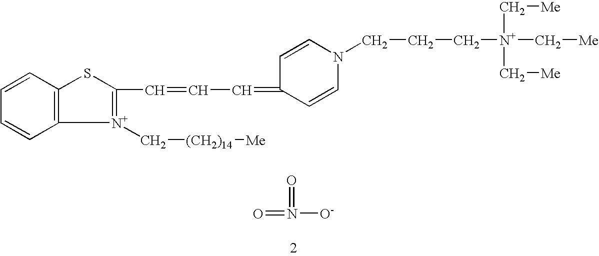
3-hexadecyl-2-[3-[1-[3-(triethylammonio)propyl]-4(1H)pyridinylidene]-1-propenyl]benzothiazolium dinitrate 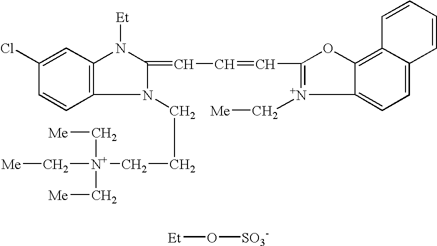
naphth[2,1-d]oxazolium, 2-[3-[5-chloro-3-ethyl-1,3-dihydro-1-[3-(triethylammonio)propyl]-2H-benzimidazol-2-ylidene]-1-propenyl]-3-ethyl-, bis(ethyl sulfate)
(sulfuric acid, monoethyl ester, on(1-),2-[3-[5-chloro-3-ethyl-1,3-dihydro-1-[3-(triethylammonio)propyl]-2H-benzimidazol-2-ylidene]-1-propenyl]-3-ethylnaphth[2,1-d]oxazolium (2:1)) 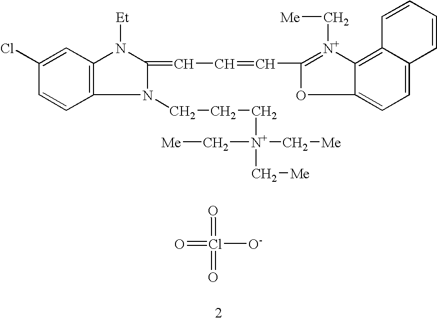
2-[3-[5-chloro-3-ethyl-1,3-dihydro-1-[3-(triethylammonio)propyl]-2H-benzimidazol-2-ylidene]-1-propenyl]-1-ethylnaphth[1,2-d]oxazolium diperchlorate 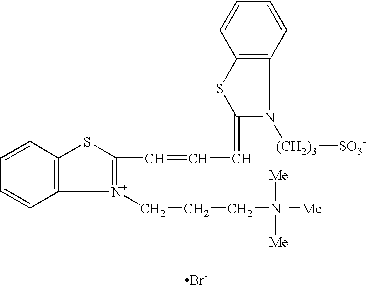
2-[3-[3-(3-sulfopropyl)-2(3H)benzothiazolylidene]-1-propenyl]-3-[3-(trimethylammonio)propyl]benzothiazolium bromide 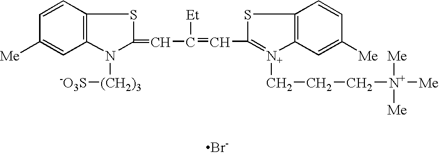
5-methyl-2-[2-[[5-methyl-3-(3-sulfopropyl)-2(3H)benzothiazolylidene]methyl]-1-butenyl]-3-[3-(trimethylammonio)propyl]benzothiazolium bromide 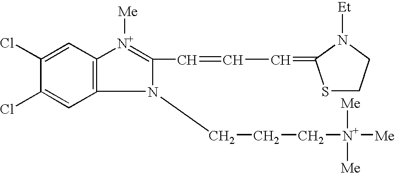
5,6-dichloro-2-[3-(3-ethyl-2-thiazolidinylidene)-1-propenyl]-1-methyl-3-[3-(trimethylammonio)propyl]-1H-benzimidazolium 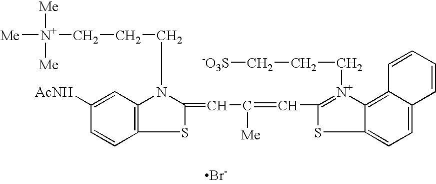
2-[3-[5-(acetylamino)-3-[3-(trimethylammonio)propyl]-2(3H)benzothiazolylidene]-2-methyl-1-propenyl]-1-(3-sulfopropyl)naphtho[1,2-d]thiazolium bromide 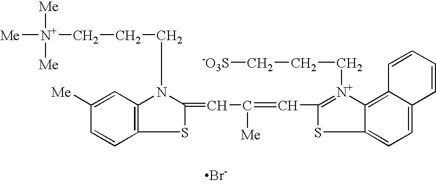
2-[2- methyl-3-[5-methyl-3-[3-(trimethylammonio)propyl]-2(3H)benzothiazolylidene]-1-propenyl]-1-(3-sulfopropyl)naphtho[1,2-d]thiazolium bromide 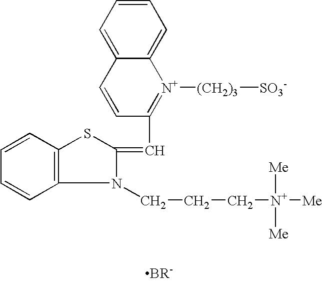
1-(3-sulfopropyl)-2-[[3-[3-(trimethylammonio)propyl]-2(3H)benzothiazolylidene]methyl]quinolinium) bromide 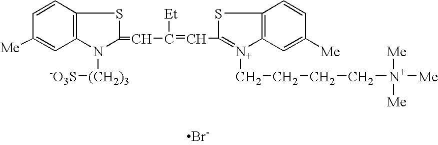
benzothiazolium, 5-methyl-2-[2-[[5-methyl-3-(3-sulfopropyl)-2(3H)-benzothiazolylidene]methyl]-1-butenyl]-3-[4-(trimethylammonio)-butyl]-, inner salt, bromide 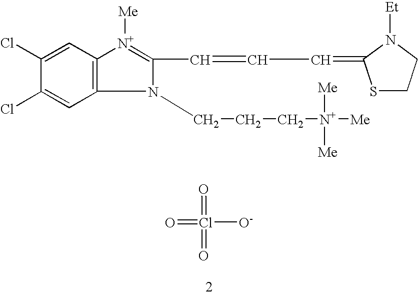
5,6-dichloro-2-[3-(3-ethyl-2-thiazolidinylidene)-1-propenyl]-1-methyl-3-[3-(trimethylammonio)propyl]-1H-benzimidazolium diperchlorate 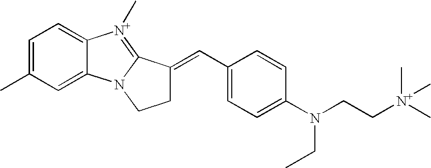
trimethyl-[2-(methyl{2-[4-(4-methyl-1,2-dihydrobenzo[d]pyrrolo[1,2-a]imidazolium-3-ylidenemethyl)phenylamino]ethyl}amino)ethyl]-ammonium 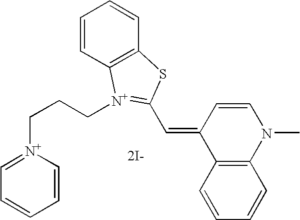
1-{3-[2-(1-methyl-1H-quinolin-4-ylidenemethyl)benzothiazolium-3-yl]propyl}pyridinium diiodide 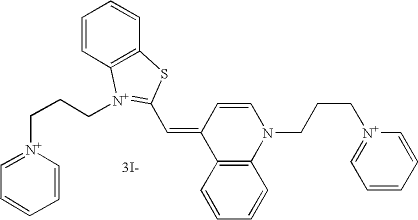
1-{3-[2-(1-(3-pyridinium-1-ylpropyl)-1H-quinolin-4-ylidene-methyl)benzothiazolium-3-yl]propyl}pyridinium triiodide 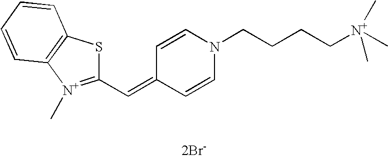
trimethyl{4-[4-(3-methylbenzothiazolium-2-ylmethylene)-4H-pyridin-1-yl]butyl}ammonium dibromide

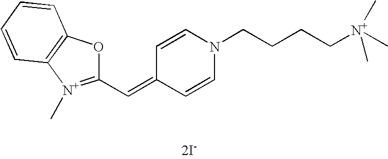

trimethyl{4-[4-(3-methylbenzoxazolium-2-ylmethylene)-4H-pyridin-1-yl]butyl}ammonium diiodide.

The composition according to the present patent application may comprise from 0.001% to 20%, preferably from 0.05% to 10% and even more preferably from 0.1% to 5% by weight of direct dye(s) of formula (I) relative to the total weight of the composition.

The dyes of the invention may be prepared according to chemical reactions that are known per se starting with chromophores bearing a cationic charge, which are capable of reacting with the chosen nitrogenous hydrocarbon-based group. The dyes may especially be prepared via the process described in document U.S. Pat. No. 3,649,162.

The dye composition in accordance with the invention may also contain one or more additional direct dyes other than the direct dyes of formula (I) described above, which may be chosen from neutral, acidic or cationic nitrobenzene dyes, neutral, acidic or cationic azo direct dyes, neutral, acidic or cationic quinone and in particular anthraquinone direct dyes, azine direct dyes, triarylmethane direct dyes, indoamine direct dyes and natural direct dyes.

Examples of additional direct dyes that may be mentioned include the cationic azo dyes described in patent applications WO-95/15144, WO-95/01772 and EP-714 954.

Among these compounds, mention may be made most particularly of the following dyes:

1,3-dimethyl-2-[[4-(dimethylamino)phenyl]azo]-1H-imidazolium chloride,

1,3-dimethyl-2-[(4-aminophenyl)azo]-1H-imidazolium chloride,

1-methyl-4-[(methylphenylhydrazono)methyl]pyridinium methyl sulfate.

Mention may also be made, among the additional azo direct dyes, of the following dyes described in the Colour Index International 3rd edition:

Disperse Red 17

Acid Yellow 9

Acid Black 1

Basic Red 22

Basic Red 76

Basic Yellow 57

Basic Brown 16

Acid Yellow 36

Acid Orange 7

Acid Red 33

Acid Red 35

Basic Brown 17

Acid Yellow 23

Acid Orange 24

Disperse Black 9.

The additional direct dye(s) preferably represent(s) from 0.001% to 20% by weight approximately and even more preferably from 0.005% to 10% by weight approximately relative to the total weight of the ready-to-use composition.

By way of example, the oxidation bases present in the composition according to the invention are chosen from phenylenediamines, bis(phenyl)alkylenediamines, para-aminophenols, ortho-aminophenols, heterocyclic bases other than the heterocyclic para-phenylenediamines of formula (I), and the addition salts thereof.

Among the para-phenylenediamines that may be mentioned, for example, are para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylene-diamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)-amino-2-methylaniline, 4-N,N-bis(β-hydroxyethyl)amino-2-chloro-aniline, 2-β-hydroxyethyl-para-phenylenediamine, 2-fluoro-para-phenylenediamine, 2-isopropyl-para-phenylenediamine, N-(β-hydroxy-propyl)-para-phenylenediamine, 2-hydroxymethyl-para-phenylene-diamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylene-diamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene and 3-hydroxy-1-(4′-aminophenyl)pyrrolidine, and the addition salts thereof with an acid.

Among the para-phenylenediamines mentioned above, para-phenylenediamine, para-tolylenediamine, 2-isopropyl-para-phenyl-enediamine, 2-β-hydroxyethyl-para-phenylenediamine, 2-β-hydroxy-ethyloxy-para-phenylenediamine, 2,6-dimethyl-para-phenylenediamine, 2,6-diethyl-para-phenylenediamine, 2,3-dimethyl-para-phenylene-diamine, N,N-bis(β-hydroxyethyl)-para-phenylenediamine, 2-chloro-para-phenylenediamine and 2-β-acetylaminoethyloxy-para-phenylene-diamine, and the addition salts thereof with an acid, are particularly preferred.

Among the bis(phenyl)alkylenediamines that may be mentioned, for example, are N,N′-bis(β-hydroxyethyl)-N,N′-bis(4′-aminophenyl)-1,3-diaminopropanol, N,N′-bis(β-hydroxyethyl)-N,N′-bis(4′-amino-phenyl)ethylenediamine, N,N′-bis(4-aminophenyl)tetramethylene-diamine, N,N′-bis(β-hydroxyethyl)-N,N′-bis(4-aminophenyl)tetra-methylenediamine, N,N′-bis(4-methylaminophenyl)tetramethylene-diamine, N,N′-bis(ethyl)-N,N′-bis(4′-amino-3′-methylphenyl)ethylene-diamine and 1,8-bis(2,5-diaminophenoxy)-3,6-dioxaoctane, and the addition salts thereof.

Among the para-aminophenols that may be mentioned, for example, are para-aminophenol, 4-amino-3-methylphenol, 4-amino-3-fluorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol and 4-amino-2-fluorophenol, and the addition salts thereof with an acid.

Among the ortho-aminophenols that may be mentioned, for example, are 2-aminophenol, 2-amino-5-methylphenol, 2-amino-6-methylphenol and 5-acetamido-2-aminophenol, and the addition salts thereof.

Among the heterocyclic bases that may be mentioned, for example, are pyridine derivatives, pyrimidine derivatives and pyrazole derivatives.

Among the pyridine derivatives that may be mentioned are the compounds described, for example, in patents GB 1 026 978 and GB 1 153 196, for instance 2,5-diaminopyridine, 2-(4-methoxyphenyl)-amino-3-aminopyridine, and 3,4-diaminopyridine, and the addition salts thereof.

Other pyridine oxidation bases that are useful in the present invention are the 3-aminopyrazolo[1,5-a]pyridine oxidation bases or addition salts thereof described, for example, in patent application FR 2 801 308. Examples that may be mentioned include pyrazolo[1,5-a]pyrid-3-ylamine, 2-acetylaminopyrazolo[1,5-a]pyrid-3-ylamine, 2-morpholin-4-ylpyrazolo[1,5-a]pyrid-3-ylamine, 3-aminopyrazolo[1,5-a]pyridine-2-carboxylic acid, 2-methoxypyrazolo[1,5-a]pyrid-3-ylamino, (3-aminopyrazolo[1,5-a]pyrid-7-yl)methanol, 2-(3-aminopyrazolo[1,5-a]pyrid-5-yl)ethanol, 2-(3-aminopyrazolo[1,5-a]pyrid-7-yl)ethanol, (3-aminopyrazolo[1,5-a]pyrid-2-yl)methanol, 3,6-diaminopyrazolo[1,5-a]pyridine, 3,4-diaminopyrazolo[1,5-a]pyridine, pyrazolo[1,5-a]-pyridine-3,7-diamine, 7-morpholin-4-ylpyrazolo[1,5-a]pyrid-3-ylamine, pyrazolo[1,5-a]pyridine-3,5-diamine, 5-morpholin-4-ylpyrazolo[1,5-a]pyrid-3-ylamine, 2-[(3-aminopyrazolo[1,5-a]pyrid-5-yl)(2-hydroxy-ethyl)amino]ethanol, 2-[(3-aminopyrazolo[1,5-a]pyrid-7-yl)(2-hydroxy-ethyl)amino]ethanol, 3-aminopyrazolo[1,5-a]pyridin-5-ol, 3-amino-pyrazolo[1,5-a]pyridin-4-ol, 3-aminopyrazolo[1,5-a]pyridin-6-ol and 3-aminopyrazolo[1,5-a]pyridin-7-ol, and the addition salts thereof.

Among the pyrimidine derivatives that may be mentioned are the compounds described, for example, in patents DE 2 359 399; JP 88-169 571; JP 05-63124; EP 0 770 375 or patent application WO 96/15765, for instance 2,4,5,6-tetraaminopyrimidine, 4-hydroxy-2,5,6-triamino-pyrimidine, 2-hydroxy-4,5,6-triaminopyrimidine, 2,4-dihydroxy-5,6-diaminopyrimidine and 2,5,6-triaminopyrimidine, and the addition salts thereof, and the tautomeric forms thereof, when a tautomeric equilibrium exists.

Among the pyrazole derivatives that may be mentioned are the compounds described in patents DE 3 843 892 and DE 4 133 957, and patent applications WO 94/08969, WO 94/08970, FR-A-2 733 749 and DE 195 43 988, for instance 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenyl-pyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxy-ethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methyl-pyrazole, 4,5-diamino-3-hydroxymethyl-1-isopropylpyrazole, 4,5-diamino-3-methyl-1-isopropylpyrazole, 4-amino-5-(2′-aminoethyl)-amino-1,3-dimethylpyrazole, 3,4,5-triaminopyrazole, 1-methyl-3,4,5-triaminopyrazole, 3,5-diamino-1-methyl-4-methylaminopyrazole and 3,5-diamino-4-(β-hydroxyethyl)amino-1-methylpyrazole, and the addition salts thereof.

The oxidation base(s) present in the composition of the invention is (are) generally present in an amount ranging from 0.001% to 20% by weight approximately, and preferably ranging from 0.005% to 6%, relative to the total weight of the dye composition.

The composition of the present invention may also comprise one or more oxidation dye precursors chosen from couplers.

The couplers may be chosen from the couplers conventionally used for dyeing keratin fibres. Among these couplers, mention may be made especially of meta-phenylenediamines, meta-diphenols, naphthalene-based couplers and heterocyclic couplers, and the addition salts thereof.

Examples that may be mentioned include 1,3-dihydroxybenzene, 1,3-dihydroxy-2-methylbenzene, 4-chloro-1,3-dihydroxybenzene, 2,4-di-amino-1-(β-hydroxyethyloxy)benzene, 2-amino-4-(β-hydroxyethyl-amino)-1-methoxybenzene, 1,3-diaminobenzene, 1,3-bis(2,4-diamino-phenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxy-benzomorpholine, 3,5-diamino-2,6-dimethoxypyridine, 1-N-(β-hydroxyethyl)amino-3,4-methylenedioxybenzene and 2,6-bis(β-hydroxyethylamino)toluene, and the addition salts thereof.

In the composition of the present invention, the coupler(s) is (are) generally present in an amount ranging from 0.001% to 20% and preferably ranging from 0.005% to 6% by weight relative to the total weight of the dye composition.

In general, the addition salts of the oxidation bases and couplers that may be used in the context of the invention are chosen especially from the addition salts with an acid, such as the hydrochlorides, hydrobromides, sulfates, citrates, succinates, tartrates, lactates, tosylates, benzenesulfonates, phosphates and acetates, and the addition salts with a base, such as sodium hydroxide, potassium hydroxide, ammonia, amines or alkanolamines.

The suitable dyeing medium, also known as the dye support, is a cosmetic medium generally consisting of water or of a mixture of water and of at least one organic solvent to dissolve the compounds that are not sufficiently water-soluble. Examples of organic solvents that may be mentioned include C₁-C₄ lower alkanols, such as ethanol and isopropanol; polyols and polyol ethers, for instance 2-butoxyethanol, propylene glycol, propylene glycol monomethyl ether, diethylene glycol monoethyl ether and monomethyl ether, and also aromatic alcohols, for instance benzyl alcohol or phenoxyethanol, and mixtures thereof.

The solvents are preferably present in proportions preferably of between 1% and 40% by weight approximately and even more preferably between 5% and 30% by weight approximately relative to the total weight of the dye composition.

The dye composition in accordance with the invention may also contain various adjuvants conventionally used in hair dye compositions, such as anionic, cationic, nonionic, amphoteric or zwitterionic surfactants or mixtures thereof, anionic, cationic, nonionic, amphoteric or zwitterionic polymers or mixtures thereof, mineral or organic thickeners, and in particular anionic, cationic, nonionic and amphoteric polymeric associative thickeners, antioxidants, penetrators, sequestering agents, fragrances, buffers, dispersants, conditioning agents, for instance volatile or non-volatile, modified or unmodified silicones, film-forming agents, ceramides, preserving agents and opacifiers.

The above adjuvants are generally present in an amount for each of between 0.01% and 20% by weight relative to the weight of the composition.

Needless to say, a person skilled in the art will take care to select this or these optional additional compound(s) such that the advantageous properties intrinsically associated with the oxidation dye composition in accordance with the invention are not, or are not substantially, adversely affected by the envisaged addition(s).

The pH of the dye composition in accordance with the invention is generally between 3 and 12 approximately and preferably between 5 and 11 approximately. It may be adjusted to the desired value by means of acidifying or basifying agents usually used for dyeing keratin fibres, or alternatively using standard buffer systems.

Among the acidifying agents that may be mentioned, for example, are mineral or organic acids such as hydrochloric acid, orthophosphoric acid, sulfuric acid, carboxylic acids, for instance acetic acid, tartaric acid, citric acid or lactic acid, and sulfonic acids.

Among the basifying agents that may be mentioned, for example, are aqueous ammonia, alkaline carbonates, alkanolamines such as monoethanolamine, diethanolamine and triethanolamine and derivatives thereof, sodium hydroxide, potassium hydroxide and the compounds of formula (II) below:

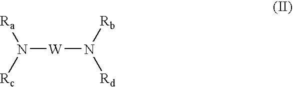

in which W is a propylene residue optionally substituted with a hydroxyl group or a C₁-C₄ alkyl radical; Rₐ, R_{b}, R_{c} and R_{d}, which may be identical or different, represent a hydrogen atom or a C₁-C₄ alkyl or C₁-C₄ hydroxyalkyl radical.

The dye composition according to the invention may be in various forms, such as in the form of liquids, creams or gels, or in any other form that is suitable for dyeing keratin fibres, and especially human hair.

The use of the process according to the present patent application includes the application of a dye composition according to the present patent application to the keratin fibres, followed by a step consisting in leaving the composition on the fibres for a period that is sufficient to dye the hair, this period generally being between 5 minutes and 1 hour and preferably between 15 minutes and 1 hour.

The process for dyeing keratin fibres may comprise a step in which an oxidizing agent is used at acidic, neutral or alkaline pH. The oxidizing agent may be added to the composition of the invention just at the time of use, or it may be used starting with an oxidizing composition containing it, which is applied simultaneously or sequentially to the composition of the invention.

This process may especially be performed when the composition according to the invention comprises at least one oxidation dye precursor.

According to one particular embodiment, the composition according to the present invention comprising at least one oxidation dye precursor is mixed, preferably at the time of use, with a composition containing, in a suitable dyeing medium, at least one oxidizing agent, this oxidizing agent being present in an amount that is sufficient to develop a coloration. The mixture obtained is then applied to the keratin fibres. After a leave-in time of from 5 minutes to 1 hour approximately and preferably 15 minutes to 1 hour approximately, the keratin fibres are rinsed, washed with a shampoo, rinsed again and then dried.

The oxidizing agents conventionally used for dyeing keratin fibres are, for example, hydrogen peroxide, urea peroxide, alkali metal bromates, persalts such as perborates and persulfates, peracids and oxidase enzymes, among which mention may be made of peroxidases, two-electron oxidoreductases such as uricases, and four-electron oxygenases, for instance laccases. Hydrogen peroxide is particularly preferred.

The oxidizing composition may also contain various adjuvants conventionally used in hair dye compositions and as defined above.

In the particular embodiment of the invention in which the dye composition is mixed with an oxidizing composition, the pH of the oxidizing composition containing the oxidizing agent is such that, after mixing with the dye composition, the pH of the resulting composition applied to the keratin fibres preferably ranges between 3 and 12 approximately, preferably between 5 and 11 approximately and even more preferably between 6 and 8.5. It may be adjusted to the desired value by means of acidifying or basifying pH regulators usually used in the dyeing of keratin fibres and as defined above.

The composition applied to the keratin fibres may be in various forms, such as in the form of liquids, creams or gels, or in any other form that is suitable for dyeing keratin fibres, and especially human hair. In particular, it may be packaged under pressure in an aerosol can in the presence of a propellant, and may form a mousse.

A subject of the present patent application is also a multi-compartment device or dyeing kit, in which a first compartment contains the dye composition defined above and a second compartment contains an oxidizing composition. This device may be equipped with a means for applying the desired mixture onto the hair, such as the devices described in patent FR-2 586 913 in the name of the Applicant.

Using this device, it is possible to dye keratin fibres via a process that includes mixing a dye composition in accordance with the invention with an oxidizing agent as defined above, and applying the mixture obtained to the keratin fibres for a time that is sufficient to develop the desired coloration.

The examples that follow are intended to illustrate the invention without, however, being limiting in nature.

### EXAMPLE 1

Hair dye composition

| Dye (1) | 2.5 × 10⁻³ | mol |
|---|---|---|
| Hydroxyethylcellulose | 1.44 | g |
| Parabens | 0.06 | g |
| Decyl glucoside | 10 | g |
| Benzyl alcohol | 8 | g |
| PEG-6 | 12 | g |
| Water qs | 100 | g |

(1) 1-{3-[2-(1-methyl-1H-quinolin-4-ylidenemethyl)-benzothiazolium-3-yl]propyl}pyridinium diiodide

After a leave-in time of 30 minutes on grey hair containing 90% white hairs and rinsing, an orange shade is obtained.

### EXAMPLE 2

Hair dye composition

| Dye (1) | 2.5 × 10⁻³ | mol |
|---|---|---|
| Hydroxyethylcellulose | 1.44 | g |
| Parabens | 0.06 | g |
| Decyl glucoside | 10 | g |
| Benzyl alcohol | 8 | g |
| PEG-6 | 12 | g |
| Water qs | 100 | g |

(1) 1-{3-[2-(1-(3-pyridinium-1-ylpropyl)-1H-quinolin-4-ylidenemethyl)benzothiazolium-3-yl]propyl}pyridinium triiodide

After a leave-in time of 30 minutes on grey hair containing 90% white hairs and rinsing, an orange-red shade is obtained.

### EXAMPLE 3

Hair dye composition

| Dye (1) | 2.5 × 10⁻³ | mol |
|---|---|---|
| Hydroxyethylcellulose | 1.44 | g |
| Parabens | 0.06 | g |
| Decyl glucoside | 10 | g |
| Benzyl alcohol | 8 | g |
| PEG-6 | 12 | g |
| Water qs | 100 | g |

(1) trimethyl{4-[4-(3-methylbenzothiazolium-2-ylmethylene)-4H-pyrid-1-yl]butyl}ammonium dibromide

After a leave-in time of 30 minutes on grey hair containing 90% white hairs and rinsing, a yellow shade is obtained.

### EXAMPLE 4

Hair dye composition

| Dye (1) | 2.5 × 10⁻³ | mol |
|---|---|---|
| Hydroxyethylcellulose | 1.44 | g |
| Parabens | 0.06 | g |
| Decyl glucoside | 10 | g |
| Benzyl alcohol | 8 | g |
| PEG-6 | 12 | g |
| Water qs | 100 | g |

(1) trimethyl{4-[4-(3-methylbenzoxazolium-2-ylmethylene)-4H-pyrid-1-yl]butyl}ammonium diiodide

After a leave-in time of 30 minutes on grey hair containing 90% white hairs and rinsing, a yellow shade is obtained.

### EXAMPLE 5

Hair dye composition

| | |
|---|---|
| Dye (1) | 0.15 g |
| Ethanol | 10 g |
| Alkyl polyglucoside | 5 g of active material |
| 2-Amino-2-methylpropanol | qs pH 8.0 |
| Water qs | 100 g |

(1) trimethyl {3-[4-(1-methylnaphtho[1,2-d]thiazolium-2-ylmethylene)-4H-pyrimidin-1-yl]propyl}ammonium acetate

### EXAMPLE 6

Hair dye composition

| | |
|---|---|
| Dye (1) | 0.15 g |
| Ethanol | 10 g |
| Alkyl polyglucoside | 5 g of active material |
| 2-Amino-2-methylpropanol | qs pH 8.0 |
| Water qs | 100 g |

(1) 3-[3-(trimethylammonio)propyl]-2-[5-[3-[3-(trimethylammonio)propyl]-2(3H)benzothiazolylidene]-1,3-pentadienyl]benzothiazolium triiodide

### EXAMPLE 7

Hair dye composition

| | |
|---|---|
| Dye (1) | 0.15 g |
| Ethanol | 10 g |
| Alkyl polyglucoside | 5 g of active material |
| 2-Amino-2-methylpropanol | qs pH 8.0 |
| Water qs | 100 g |

(1) 3-methyl-2-[5-[1-[3-(trimethylammonio)propyl]-4(1H)pyridinylidene]-1,3-pentadienyl]benzoxazolium diiodide

1. Use, as direct dye in compositions for dyeing keratin fibres, in particular human keratin fibres such as the hair, or for the manufacture of such compositions, of a compound of formula (I) 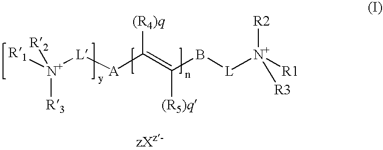
in which:
y denotes 0 or 1;
n denotes an integer ranging from 1 to 4;
q and q′ denote, independently of each other, 0 or 1;
X^{z′} denotes an organic or mineral anion;
z and z′ are such that the overall charge of the molecule is zero;
only one or both of the extreme carbon atoms of the group 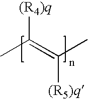
possibly forming part of the rings A and/or B;
A and/or B denotes(s), independently of each other, an aromatic non-heterocyclic cyclic group or a 5- to 15-membered, substituted of unsubstituted heterocyclic group, at least one of the groups A or B bearing at least one positive charge on condition that, if A is a non-heterocyclic ring, then B denotes a benzothiazolium, naphthothiazolium, naphthoxazolium, benzoxazolium, benzimidazolium, thiazolium, pyrimidobenzimidazolium, benzopyrroloimidazolium, pyrido benzoxazolium, pyrido benzothiazolium, pyrrolo benzoxazolium, pyrrolo benzothiazolium, naphthothiazolo pyridinium, azepino thiazolium, diazepino benzoxazolium, azepino benzoxazolium, pyrido benzoxazolium, azocino benzoxazolium, azocino benzothiazolium, azepino benzothiazolium or quinolinium group;
L and L′ denote, independently of each other, a linear, branched or cyclic alkylene chain containing from 1 to 30 carbon atoms and/or a substituted or unsubstituted aromatic chain containing from 6 to 30 carbon atoms; the chain possibly being interrupted or ending with one or more heteroaromatic groups chosen from O, S, NH and NR where R represents an optionally substituted, linear or branched C1-C20 alkyl radical, or interrupted with one or more heterocyclic or non-heterocyclic, aromatic or non-aromatic cyclic radicals, the chain itself possibly bearing one or more quaternary cationic charges and one or more carbon atoms of the chain possibly being replaced with a carbonyl group;
R1, R2, R3, R′1, R′2 and R′3 denote, independently of each other, a linear, branched or cyclic, optionally substituted C1-C20 alkyl radical, two or more of these radicals possibly forming, with the nitrogen atom bearing them and optionally one of the carbon or nitrogen atoms of L for R1, R2 and/or R3 or L′ for R′1, R′2 and/or R′3, an optionally substituted saturated, unsaturated or aromatic heterocycle that may contain one or more additional hetero atoms; an optionally substituted C6-C30 aryl radical;
R4 and R5 denote, independently of each other, a hydrogen atom, a halogen atom, an optionally substituted C6-C30 aryl radical, or a linear, branched or cyclic, optionally substituted C1-C10 alkyl radical; the radicals R4 and R5 may form with the carbon atoms bearing them a 5- to 10-membered ring.

2. Dye composition for dyeing keratin fibres, in particular human keratin fibres such as the hair, comprising, in a suitable dyeing medium, at least one oxidation base and at least one compound of formula (I) as defined in claim 1.

3. Composition according to claim 2, characterized in that it comprises from 0.001% to 20%, preferably from 0.05% to 10% and even more preferably from 0.1% to 5% by weight of direct dye(s) of formula (I) relative to the total weight of the composition.

4. Dye composition according to claim 2 or 3, characterized in that the oxidation base is chosen from para-phenylenediamines, bis(phenyl)alkylenediamines, para-aminophenols, ortho-aminophenols and heterocyclic bases, and the addition salts thereof.

5. Dye composition according to any one of claims 2 to 4, characterized in that the oxidation base(s) is (are) present in an amount of between 0.001% and 20% by weight and preferably between 0.005% and 6% by weight relative to the total weight of the composition.

6. Composition according to one of claims 2 to 5, characterized in that it comprises at least one additional direct dye.

7. Composition according to one of claims 2 to 6, characterized in that it contains at least one oxidation dye precursor chosen from couplers.

8. Composition according to claim 7, characterized in that the coupler is chosen from meta-phenylenediamines, meta-amino-phenols, meta-diphenols, naphthalene-based couplers and heterocyclic couplers, and the addition salts thereof.

9. Composition according to claim 7, characterized in that the coupler is chosen from 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene and 2,6-bis(β-hydroxyethylamino)toluene, and the addition salts thereof.

10. Composition according to one of claims 7 to 9, characterized in that the coupler(s) is (are) present in an amount of between 0.001% and 20% and preferably between 0.005% and 6% by weight relative to the total weight of the composition.

11. Composition according to one of claims 2 to 10, characterized in that it comprises at least one solvent.

12. Composition according to claim 11, characterized in that the solvent is chosen from ethanol, propylene glycol, glycerol and polyol monoethers.

13. Composition according to one of claims 2 to 12, characterized in that it comprises at least one adjuvant chosen from anionic, cationic, nonionic, amphoteric or zwitterionic surfactants or mixtures thereof, anionic, cationic, nonionic, amphoteric or zwitterionic polymers or mixtures thereof, mineral or organic thickeners, antioxidants, penetrators, sequestering agents, fragrances, buffers, dispersants, conditioning agents, film-forming agents, ceramides, preserving agents and opacifiers.

14. Composition according to one of claims 2 to 13, characterized in that it comprises at least one oxidizing agent chosen from hydrogen peroxide, urea peroxide, alkali metal bromates, persalts, peracids and oxidase enzymes.

15. Composition according to claim 14, characterized in that the oxidizing agent is hydrogen peroxide.

16. Process for dyeing keratin fibres, characterized in that it includes the application of a composition according to one of claims 2 to 15 to the keratin fibres, followed by a step consisting in leaving the composition on the fibres for a period of between 5 minutes and one hour and preferably between 15 minutes and one hour.

17. Use of a composition according to one of claims 2 to 15 for dyeing keratin fibres, in particular human keratin fibres such as the hair.

18. Use of a composition according to one of claims 2 to 15 on keratin fibres, in particular human keratin fibres such as the hair, to obtain dyeing results that show good resistance to external agents and to shampoo.
